(19) United States
(12) Patent Application Publication
(10) Pub. No.: US 2016/0237445 A1
(43) Pub. Date: Aug. 18, 2016

(54) SOYBEAN PIP1 PROMOTER AND ITS USE IN CONSTITUTIVE EXPRESSION OF TRANSGENIC GENES IN PLANTS

(71) Applicant: E. I. DU PONT DE NEMOURS AND COMPANY, Wilmington, DE (US)

(72) Inventor: Zhongsen Li, Hockessin, DE (US)

(21) Appl. No.: 15/030,246

(22) PCT Filed: Oct. 17, 2014
(86) PCT No.: PCT/US2014/061062

§ 371 Date: Apr. 18, 2016

Related U.S. Application Data
(60) Provisional application No. 61/893,358, filed on Oct. 21, 2013.

(51) Int. Cl. C12N 15/82

(52) U.S. Cl. CPC C12N 15/8216; C12N 15/8279; C12N 15/8274; C12N 15/8286; C12N 15/8245; C12N 15/8247; C12N 15/8251; C12N 15/8273

(57) ABSTRACT

The disclosure relates to gene expression regulatory sequences from soybean, specifically to recombinant DNA constructs comprising the promoter of a soybean plasma membrane intrinsic protein gene and fragments thereof and their use in promoting the expression of one or more heterologous nucleic acid fragments in a constitutive manner in plants. The disclosure further discloses compositions, polynucleotide constructs, transformed host cells, transgenic plants and seeds containing the recombinant construct with the promoter, and methods for preparing and using the same.

This application claims the benefit of U.S. Patent Application Ser. No. 61/893,358, filed Oct. 21, 2013, which is herein incorporated by reference in its entirety.

### FIELD

This disclosure relates to a plant promoter GM-PIP1 and fragments thereof and their use in altering expression of at least one heterologous nucleotide sequence in plants in a tissue-independent or constitutive manner.

### REFERENCE TO SEQUENCE LISTING SUBMITTED ELECTRONICALLY

The official copy of the sequence listing is submitted electronically via EFS-Web as an ASCII formatted sequence listing with a file named 20141017_BB2118PCT_SequenceListing created on Oct. 17, 2014 and having a size of 74 kilobytes and is filed concurrently with the specification. The sequence listing contained in this ASCII formatted document is part of the specification and is herein incorporated by reference in its entirety.

### BACKGROUND

Recent advances in plant genetic engineering have opened new doors to engineer plants to have improved characteristics or traits, such as plant disease resistance, insect resistance, herbicidal resistance, yield improvement, improvement of the nutritional quality of the edible portions of the plant, and enhanced stability or shelf-life of the ultimate consumer product obtained from the plants. Thus, a desired gene (or genes) with the molecular function to impart different or improved characteristics or qualities, can be incorporated properly into the plant's genome. The newly integrated gene (or genes) coding sequence can then be expressed in the plant cell to exhibit the desired new trait or characteristics. It is important that appropriate regulatory signals must be present in proper configurations in order to obtain the expression of the newly inserted gene coding sequence in the plant cell. These regulatory signals typically include a promoter region, a 5′ non-translated leader sequence and a 3′ transcription termination/polyadenylation sequence.

A promoter is a non-coding genomic DNA sequence, usually upstream (5′) to the relevant coding sequence, to which RNA polymerase binds before initiating transcription. This binding aligns the RNA polymerase so that transcription will initiate at a specific transcription initiation site. The nucleotide sequence of the promoter determines the nature of the RNA polymerase binding and other related protein factors that attach to the RNA polymerase and/or promoter, and the rate of RNA synthesis. The RNA is processed to produce messenger RNA (mRNA) which serves as a template for translation of the RNA sequence into the amino acid sequence of the encoded polypeptide. The 5′ non-translated leader sequence is a region of the mRNA upstream of the coding region that may play a role in initiation and translation of the mRNA. The 3′ transcription termination/polyadenylation signal is a non-translated region downstream of the coding region that functions in the plant cell to cause termination of the RNA synthesis and the addition of polyadenylate nucleotides to the 3′ end.

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters” if the promoters direct RNA synthesis preferably in certain tissues but also in other tissues at reduced levels. Since patterns of expression of a chimeric gene (or genes) introduced into a plant are controlled using promoters, there is an ongoing interest in the isolation of novel promoters which are capable of controlling the expression of a chimeric gene or (genes) at certain levels in specific tissue types or at specific plant developmental stages.

Certain promoters are able to direct RNA synthesis at relatively similar levels across all tissues of a plant. These are called “constitutive promoters” or “tissue-independent” promoters. Constitutive promoters can be divided into strong, moderate and weak according to their effectiveness to direct RNA synthesis. Since it is necessary in many cases to simultaneously express a chimeric gene (or genes) in different tissues of a plant to get the desired functions of the gene (or genes), constitutive promoters are especially useful in this consideration. Though many constitutive promoters have been discovered from plants and plant viruses and characterized, there is still an ongoing interest in the isolation of more novel constitutive promoters which are capable of controlling the expression of a chimeric gene or (genes) at different levels and the expression of multiple genes in the same transgenic plant for gene stacking.

### SUMMARY OF THE DISCLOSURE

This disclosure concerns a recombinant DNA construct comprising at least one heterologous nucleotide sequence operably linked to a promoter wherein said promoter comprises the nucleotide sequence set forth in SEQ ID NOs: 1, 2, 3, 4, 5, 6, or 49, or said promoter comprises a functional fragment of the nucleotide sequence set forth in SEQ ID NOs: 1, 2, 3, 4, 5, 6, or 39, or wherein said promoter comprises a nucleotide sequence having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity, based on the Clustal V method of alignment with pairwise alignment default parameters (KTUPLE=2, GAP PENALTY=5, WINDOW=4 and DIAGONALS SAVED=4), when compared to the nucleotide sequence of SEQ ID NO:1, 2, 3, 4, 5, 6, or 49.

In another embodiment, this disclosure concerns a recombinant DNA construct comprising a nucleotide sequence comprising any of the sequences set forth in SEQ ID NO:1, SEQ ID NO:2, SEQ ID NO:3, SEQ ID NO:4, SEQ ID NO:5, SEQ ID NO:6, or SEQ ID NO:49, or a functional fragment thereof, operably linked to at least one heterologous sequence, wherein said nucleotide sequence is a constitutive promoter.

In another embodiment, this disclosure concerns a recombinant DNA construct comprising a nucleotide sequence having at least 95% identity, based on the Clustal V method of alignment with pairwise alignment default parameters (KTUPLE=2, GAP PENALTY=5, WINDOW=4 and DIAGONALS SAVED=4), when compared to the sequence set forth in SEQ ID NO:6.

In another embodiment, the disclosure concerns an isolated polynucleotide comprising a promoter region of the plasma membrane intrinsic protein (PIP1) Glycine max gene as set forth in SEQ ID NO:1, wherein said promoter comprises a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 100 6, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 11511, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 12312, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 13013, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362 or 1363 consecutive nucleotides, wherein the first nucleotide deleted is the cytosine nucleotide [‘C’] at position 1 of SEQ ID NO:1. This disclosure also concerns an isolated polynucleotide of the embodiments disclosed herein, wherein the polynucleotide is a constitutive promoter.

In one embodiment, this disclosure concerns a recombinant DNA construct comprising at least one heterologous nucleotide sequence operably linked to the promoter of the disclosure.

In one embodiment, this disclosure concerns a cell, plant, or seed comprising a recombinant DNA construct of the present disclosure.

In one embodiment, this disclosure concerns plants comprising this recombinant DNA construct and seeds obtained from such plants.

In one embodiment, this disclosure concerns a method of altering (increasing or decreasing) expression of at least one heterologous nucleic acid fragment in a plant cell which comprises:
(a) transforming a plant cell with the recombinant expression construct described above;
(b) growing fertile mature plants from the transformed plant cell of step (a);
(c) selecting plants containing the transformed plant cell wherein the expression of the heterologous nucleic acid fragment is increased or decreased.

In one embodiment, this disclosure concerns a method for expressing a yellow fluorescent protein ZS-YELLOW1 N1 (YFP) in a host cell comprising:
(a) transforming a host cell with a recombinant expression construct comprising at least one ZS-YELLOW1 N1 nucleic acid fragment operably linked to a promoter wherein said promoter consists essentially of the nucleotide sequence set forth in SEQ ID NOs:1, 2, 3, 4, 5, 6, 7 or 49; and
(b) growing the transformed host cell under conditions that are suitable for expression of the recombinant DNA construct, wherein expression of the recombinant DNA construct results in production of increased levels of ZS-YELLOW1 N1 protein in the transformed host cell when compared to a corresponding nontransformed host cell.

In one embodiment, this disclosure concerns an isolated nucleic acid fragment comprising a plant plasma membrane intrinsic protein (PIP1) gene promoter.

In one embodiment, this disclosure concerns a method of altering a marketable plant trait. The marketable plant trait concerns genes and proteins involved in disease resistance, herbicide resistance, insect resistance, carbohydrate metabolism, fatty acid metabolism, amino acid metabolism, plant development, plant growth regulation, yield improvement, drought resistance, cold resistance, heat resistance, and salt resistance.

In one embodiment, this disclosure concerns an isolated polynucleotide linked to a heterologous nucleotide sequence. The heterologous nucleotide sequence encodes a protein involved in disease resistance, herbicide resistance, insect resistance; carbohydrate metabolism, fatty acid metabolism, amino acid metabolism, plant development, plant growth regulation, yield improvement, drought resistance, cold resistance, heat resistance, or salt resistance in plants.

### BRIEF DESCRIPTION OF THE DRAWINGS AND SEQUENCE LISTINGS

The patent or application file contains at least one drawing executed in color. Copies of this patent or application publication with color drawing(s) will be provided by the Office upon request and payment of necessary fee.

The disclosure can be more fully understood from the following detailed description and the accompanying drawings and Sequence Listing that form a part of this application.

FIG. 1 is the logarithm of relative quantifications of the soybean plasma membrane intrinsic protein gene (PSO332986) expression in 14 soybean tissues by quantitative RT-PCR. The gene expression profile indicates that the PIP1 gene is moderately expressed in all the checked tissues.

FIG. 2 is the relative expression of the soybean plasma membrane intrinsic protein (PIP1) gene (Glyma14g06680.1) in twenty soybean tissues by Illumina (Solexa) digital gene expression dual-tag-based mRNA profiling. The gene expression profile indicates that the PIP1 gene is expressed in all the checked tissues.

FIG. 3A-3B shows the PIP1 promoter copy number analysis by Southern. FIG. 3A shows the Southern Blot with restriction enzymes listed on top. FIG. 3B show a diagram of the promoter and location of the DraI restriction sites and 690 bp probe.

FIG. 4 shows the schematic description of the full length construct QC386 and its progressive truncation constructs, QC386-1Y, QC386-2Y, QC386-3Y, QC386-4Y, QC386-5Y, and QC386-6Y, of the PIP1 promoter. The size of each promoter is given at the left end of each drawing. QC386-1Y has 1584 bp of the 1592 bp PIP1 promoter in QC386 with the NcoI site removed and like the other deletion constructs with the attB site between the promoter and ZS-YELLOW N1 reporter gene.

The sequence descriptions summarize the Sequence Listing attached hereto. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219(2):345-373 (1984).

SEQ ID NO:1 is a 1592 bp (base pair) DNA sequence comprising the full length soybean PIP1 promoter flanked by Xma1 (cccggg) and NcoI (ccatgg) restriction sites.

SEQ ID NO:2 is a 1584 bp full length form of the PIP1 promoter shown in SEQ ID NO:1 (bp 4-1587 of SEQ ID NO:1) with the 5′ XmaI and 3′ end NcoI sites removed.

SEQ ID NO:3 is a 1258 bp truncated form of the PIP1 promoter shown in SEQ ID NO:1 (bp 330-1587 of SEQ ID NO:1).

SEQ ID NO:4 is a 1002 bp truncated form of the PIP1 promoter shown in SEQ ID NO:1 (bp 586-1587 of SEQ ID NO:1).

SEQ ID NO:5 is a 690 bp truncated form of the PIP1 promoter shown in SEQ ID NO:1 (bp 898-1587 of SEQ ID NO:1).

SEQ ID NO:6 is a 448 bp truncated form of the PIP1 promoter shown in SEQ ID NO:1 (bp 1140-1587 of SEQ ID NO:1).

SEQ ID NO:7 is a 229 bp truncated form of the PIP1 promoter shown in SEQ ID NO:1 (bp 1359-1587 of SEQ ID NO:1).

SEQ ID NO:8 is an oligonucleotide primer used as a gene-specific sense primer in the PCR amplification of the full length PIP1 promoter in SEQ ID NO:1 when paired with SEQ ID NO:9. A restriction enzyme XmaI recognition site CCCGGG is included for subsequent cloning.

SEQ ID NO:9 is an oligonucleotide primer used as a gene-specific antisense anchor primer in the PCR amplification of the full length PIP1 promoter in SEQ ID NO:1 when paired with SEQ ID NO:8. A restriction enzyme NcoI recognition site CCATGG is included for subsequent cloning.

SEQ ID NO:10 is an oligonucleotide primer used as an antisense primer in the PCR amplifications of the truncated PIP1 promoters in SEQ ID NOs:2, 3, 4, 5, 6, or 7 when paired with SEQ ID NOs: 11, 12, 13, 14, 15, or 16, respectively.

SEQ ID NO:11 is an oligonucleotide primer used as a sense primer in the PCR amplification of the full length PIP1 promoter in SEQ ID NO:2 when paired with SEQ ID NO:10.

SEQ ID NO:12 is an oligonucleotide primer used as a sense primer in the PCR amplification of the truncated PIP1 promoter in SEQ ID NO:3 when paired with SEQ ID NO:10.

SEQ ID NO:13 is an oligonucleotide primer used as a sense primer in the PCR amplification of the truncated PIP1 promoter in SEQ ID NO:4 when paired with SEQ ID NO:10.

SEQ ID NO:14 is an oligonucleotide primer used as a sense primer in the PCR amplification of the truncated PIP1 promoter in SEQ ID NO:5 when paired with SEQ ID NO:10.

SEQ ID NO:15 is an oligonucleotide primer used as a sense primer in the PCR amplification of the truncated PIP1 promoter in SEQ ID NO:6 when paired with SEQ ID NO:10.

SEQ ID NO:16 is an oligonucleotide primer used as a sense primer in the PCR amplification of the truncated PIP1 promoter in SEQ ID NO:7 when paired with SEQ ID NO:10.

SEQ ID NO:17 is the 1247 bp nucleotide sequence of the putative soybean plasma membrane intrinsic protein gene PIP1 (PSO332986). Nucleotides 1 to 67 are the 5′ untranslated sequence, nucleotides 68 to 70 are the translation initiation codon, nucleotides 68 to 934 are the polypeptide coding region, nucleotides 935 to 937 are the termination codon, and nucleotides 938 to 1247 are part of the 3′ untranslated sequence.

SEQ ID NO:18 is the predicted 289 aa (amino acid) long peptide sequence translated from the coding region of the putative soybean plasma membrane intrinsic protein gene PIP1 nucleotide sequence SEQ ID NO:17.

SEQ ID NO:19 is the 4869 bp sequence of plasmid QC386.

SEQ ID NO:20 is the 8409 bp sequence of plasmid QC324i.

SEQ ID NO:21 is the 9394 bp sequence of plasmid QC389.

SEQ ID NO:22 is the 4401 bp sequence of plasmid QC386-1.

SEQ ID NO:23 is the 5286 bp sequence of plasmid QC330.

SEQ ID NO:24 is the 5242 bp sequence of plasmid QC386-1Y.

SEQ ID NO:25 is an oligonucleotide primer used in the diagnostic PCR to check for soybean genomic DNA presence in total RNA or cDNA when paired with SEQ ID NO:26.

SEQ ID NO:26 is an oligonucleotide primer used in the diagnostic PCR to check for soybean genomic DNA presence in total RNA or cDNA when paired with SEQ ID NO:25.

SEQ ID NO:27 is a sense primer used in quantitative RT-PCR analysis of PSO332986 gene expression.

SEQ ID NO:28 is an antisense primer used in quantitative RT-PCR analysis of PSO332986 gene expression.

SEQ ID NO:29 is a sense primer used as an endogenous control gene primer in quantitative RT-PCR analysis of gene expression.

SEQ ID NO:30 is an antisense primer used as an endogenous control gene primer in quantitative RT-PCR analysis of gene expression.

SEQ ID NO:31 is a sense primer used in the identification of BAC clones corresponding to PSO332986 gene.

SEQ ID NO:32 is an antisense primer used in the identification of BAC clones corresponding to PSO332986 gene.

SEQ ID NO:33 is a sense primer used in quantitative PCR analysis of SAMS:ALS transgene copy numbers.

SEQ ID NO:34 is a FAM labeled fluorescent DNA oligo probe used in quantitative PCR analysis of SAMS:ALS transgene copy numbers.

SEQ ID NO:35 is an antisense primer used in quantitative PCR analysis of SAMS:ALS transgene copy numbers.

SEQ ID NO:36 is a sense primer used in quantitative PCR analysis of GM-PIP1:YFP transgene copy numbers.

SEQ ID NO:37 is a FAM labeled fluorescent DNA oligo probe used in quantitative PCR analysis of GM-PIP1:YFP transgene copy numbers.

SEQ ID NO:38 is an antisense primer used in quantitative PCR analysis of GM-PIP1:YFP transgene copy numbers.

SEQ ID NO:39 is a sense primer used as an endogenous control gene primer in quantitative PCR analysis of transgene copy numbers.

SEQ ID NO:40 is a VIC labeled DNA oligo probe used as an endogenous control gene probe in quantitative PCR analysis of transgene copy numbers.

SEQ ID NO:41 is an antisense primer used as an endogenous control gene primer in quantitative PCR analysis of transgene copy numbers.

SEQ ID NO:42 is the recombination site attL1 sequence in the GATEWAY® cloning system (Invitrogen, Carlsbad, Calif.).

SEQ ID NO:43 is the recombination site attL2 sequence in the GATEWAY® cloning system (Invitrogen).

SEQ ID NO:44 is the recombination site attR1 sequence in the GATEWAY® cloning system (Invitrogen).

SEQ ID NO:45 is the recombination site attR2 sequence in the GATEWAY® cloning system (Invitrogen).

SEQ ID NO:46 is the recombination site attB1 sequence in the GATEWAY® cloning system (Invitrogen).

SEQ ID NO:47 is the recombination site attB2 sequence in the GATEWAY® cloning system (Invitrogen).

SEQ ID NO:48 is the 1378 bp nucleotide sequence of the Glycine max cDNA clone GMFLO1-52-M05 (NCBI Accession AK246127.1) containing 1247 bp sequences identical to the PIP1 gene sequence SEQ ID NO:17.

SEQ ID NO:49 is a 1591 bp fragment of native soybean genomic DNA Gm14:4892283 . . . 4893874 from cultivar “Williams” (Schmutz J. et al. Nature 463: 178-183, 2010). A nucleotide alignment of SEQ ID NO: 1, comprising the PIP1 promoter of the disclosure, and SEQ ID NO: 49 revealed a 99.7% sequence identity between the PIP1 promoter of SEQ ID NO:1 and the corresponding native soybean genomic DNA of SEQ ID NO:49, based on the Clustal V method of alignment with pairwise alignment default parameters (KTUPLE=2, GAP PENALTY=5, WINDOW=4 and DIAGONALS SAVED=4).

SEQ ID NO:50 is a 65 bp fragment of the 5′ untranslated region of the PIP promoter.

### DETAILED DESCRIPTION OF THE DISCLOSURE

The disclosure of all patents, patent applications, and publications cited herein are incorporated by reference in their entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

In the context of this disclosure, a number of terms shall be utilized.

An “isolated polynucleotide” refers to a polymer of ribonucleotides (RNA) or deoxyribonucleotides (DNA) that is single- or double-stranded, optionally containing synthetic, non-natural or altered nucleotide bases. An isolated polynucleotide in the form of DNA may be comprised of one or more segments of cDNA, genomic DNA or synthetic DNA.

The terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

A “soybean PIP1 promoter”, “GM-PIP1 promoter” or “PIP1 promoter” are used interchangeably herein, and refer to the promoter of a putative Glycine max gene with significant homology to plasma membrane intrinsic protein (PIP) genes identified in various plant species including soybean that are deposited in National Center for Biotechnology Information (NCBI) database. The term “soybean PIP1 promoter” encompasses both a native soybean promoter and an engineered sequence comprising a fragment of the native soybean promoter with a DNA linker attached to facilitate cloning. A DNA linker may comprise a restriction enzyme site.

“Promoter” refers to a nucleic acid fragment capable of controlling transcription of another nucleic acid fragment. A promoter is capable of controlling the expression of a coding sequence or functional RNA. Functional RNA includes, but is not limited to, transfer RNA (tRNA) and ribosomal RNA (rRNA). The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. New promoters of various types useful in plant cells are constantly being discovered; numerous examples may be found in the compilation by Okamuro and Goldberg (Biochemistry of Plants 15:1-82 (1989)). It is further recognized that since in most cases the exact boundaries of regulatory sequences have not been completely defined, DNA fragments of some variation may have identical promoter activity.

“Promoter functional in a plant” is a promoter capable of controlling transcription in plant cells whether or not its origin is from a plant cell.

“Tissue-specific promoter” and “tissue-preferred promoter” are used interchangeably to refer to a promoter that is expressed predominantly but not necessarily exclusively in one tissue or organ, but that may also be expressed in one specific cell.

“Developmentally regulated promoter” refers to a promoter whose activity is determined by developmental events.

“Constitutive promoter” refers to promoters active in all or most tissues or cell types of a plant at all or most developing stages. As with other promoters classified as “constitutive” (e.g. ubiquitin), some variation in absolute levels of expression can exist among different tissues or stages. The term “constitutive promoter” or “tissue-independent” are used interchangeably herein.

The promoter nucleotide sequences and methods disclosed herein are useful in regulating constitutive expression of any heterologous nucleotide sequences in a host plant in order to alter the phenotype of a plant.

A “heterologous nucleotide sequence” refers to a sequence that is not naturally occurring with the plant promoter sequence of the disclosure. While this nucleotide sequence is heterologous to the promoter sequence, it may be homologous, or native, or heterologous, or foreign, to the plant host. However, it is recognized that the instant promoters may be used with their native coding sequences to increase or decrease expression resulting in a change in phenotype in the transformed seed. The terms “heterologous nucleotide sequence”, “heterologous sequence”, “heterologous nucleic acid fragment”, and “heterologous nucleic acid sequence” are used interchangeably herein.

Among the most commonly used promoters are the nopaline synthase (NOS) promoter (Ebert et al., Proc. Natl. Acad. Sci. U.S.A. 84:5745-5749 (1987)), the octapine synthase (OCS) promoter, caulimovirus promoters such as the cauliflower mosaic virus (CaMV) 19S promoter (Lawton et al., Plant Mol. Biol. 9:315-324 (1987)), the CaMV 35S promoter (Odell et al., Nature 313:810-812 (1985)), and the figwort mosaic virus 35S promoter (Sanger et al., Plant Mol. Biol. 14:433-43 (1990)), the light inducible promoter from the small subunit of rubisco, the Adh promoter (Walker et al., Proc. Natl. Acad. Sci. U.S.A. 84:6624-66280 (1987), the sucrose synthase promoter (Yang et al., Proc. Natl. Acad. Sci. U.S.A. 87:4144-4148 (1990)), the R gene complex promoter (Chandler et al., Plant Cell 1:1175-1183 (1989)), the chlorophyll a/b binding protein gene promoter, etc. Other commonly used promoters are, the promoters for the potato tuber ADPGPP genes, the sucrose synthase promoter, the granule bound starch synthase promoter, the glutelin gene promoter, the maize waxy promoter, Brittle gene promoter, and Shrunken 2 promoter, the acid chitinase gene promoter, and the zein gene promoters (15 kD, 16 kD, 19 kD, 22 kD, and 27 kD; Perdersen et al., Cell 29:1015-1026 (1982)). A plethora of promoters is described in PCT Publication No. WO 00/18963 published on Apr. 6, 2000, the disclosure of which is hereby incorporated by reference.

The present disclosure encompasses recombinant DNA constructs comprising functional fragments of the promoter sequences disclosed herein.

A “functional fragment” refer to a portion or subsequence of the promoter sequence of the present disclosure in which the ability to initiate transcription or drive gene expression (such as to produce a certain phenotype) is retained. Fragments can be obtained via methods such as site-directed mutagenesis and synthetic construction. As with the provided promoter sequences described herein, the functional fragments operate to promote the expression of an operably linked heterologous nucleotide sequence, forming a recombinant DNA construct (also, a chimeric gene). For example, the fragment can be used in the design of recombinant DNA constructs to produce the desired phenotype in a transformed plant. Recombinant DNA constructs can be designed for use in co-suppression or antisense by linking a promoter fragment in the appropriate orientation relative to a heterologous nucleotide sequence.

A nucleic acid fragment that is functionally equivalent to the promoter of the present disclosure is any nucleic acid fragment that is capable of controlling the expression of a coding sequence or functional RNA in a similar manner to the promoter of the present disclosure.

In an embodiment of the present disclosure, the promoters disclosed herein can be modified. Those skilled in the art can create promoters that have variations in the polynucleotide sequence. The polynucleotide sequence of the promoters of the present disclosure as shown in SEQ ID NOS: 1, 2, 3, 4, 5, 6, 7, and 49, may be modified or altered to enhance their control characteristics. As one of ordinary skill in the art will appreciate, modification or alteration of the promoter sequence can also be made without substantially affecting the promoter function. The methods are well known to those of skill in the art. Sequences can be modified, for example by insertion, deletion, or replacement of template sequences in a PCR-based DNA modification approach.

A “variant promoter”, as used herein, is the sequence of the promoter or the sequence of a functional fragment of a promoter containing changes in which one or more nucleotides of the original sequence is deleted, added, and/or substituted, while substantially maintaining promoter function. One or more base pairs can be inserted, deleted, or substituted internally to a promoter. In the case of a promoter fragment, variant promoters can include changes affecting the transcription of a minimal promoter to which it is operably linked. Variant promoters can be produced, for example, by standard DNA mutagenesis techniques or by chemically synthesizing the variant promoter or a portion thereof.

Methods for construction of chimeric and variant promoters of the present disclosure include, but are not limited to, combining control elements of different promoters or duplicating portions or regions of a promoter (see for example, U.S. Pat. No. 4,990,607; U.S. Pat. No. 5,110,732; and U.S. Pat. No. 5,097,025). Those of skill in the art are familiar with the standard resource materials that describe specific conditions and procedures for the construction, manipulation, and isolation of macromolecules (e.g., polynucleotide molecules and plasmids), as well as the generation of recombinant organisms and the screening and isolation of polynucleotide molecules.

In some aspects of the present disclosure, the promoter fragments can comprise at least about 20 contiguous nucleotides, or at least about 50 contiguous nucleotides, or at least about 75 contiguous nucleotides, or at least about 100 contiguous nucleotides, or at least about 150 contiguous nucleotides, or at least about 200 contiguous nucleotides of SEQ ID NO:1, SEQ ID NO:2, SEQ ID NO:3, SEQ ID NO:4, SEQ ID NO:5, SEQ ID NO:6, SEQ ID NO:7 or SEQ ID NO:49. In another aspect of the present disclosure, the promoter fragments can comprise at least about 250 contiguous nucleotides, or at least about 300 contiguous nucleotides, or at least about 350 contiguous nucleotides, or at least about 400 contiguous nucleotides, or at least about 450 contiguous nucleotides, or at least about 500 contiguous nucleotides, or at least about 550 contiguous nucleotides, or at least about 600 contiguous nucleotides, or at least about 650 contiguous nucleotides, or at least about 700 contiguous nucleotides, or at least about 750 contiguous nucleotides, or at least about 800 contiguous nucleotides, or at least about 850 contiguous nucleotides, or at least about 900 contiguous nucleotides or at least about 950 contiguous nucleotides, or at least about 1000 contiguous nucleotides, or at least about 1050 contiguous nucleotides, or at least about 1100 contiguous nucleotides, or at least about 1150 contiguous nucleotides, or at least about 1200 contiguous nucleotides, or at least about 1250 contiguous nucleotides, or at least about 1300 contiguous nucleotides, or at least about 1350 contiguous nucleotides of SEQ ID NO:1. In another aspect, a promoter fragment is the nucleotide sequence set forth in SEQ ID NO:2, SEQ ID NO:3, SEQ ID NO:4, SEQ ID NO:5, SEQ ID NO:6, SEQ ID NO:7 or SEQ ID NO:49. The nucleotides of such fragments will usually comprise the TATA recognition sequence of the particular promoter sequence. Such fragments may be obtained by use of restriction enzymes to cleave the naturally occurring promoter nucleotide sequences disclosed herein, by synthesizing a nucleotide sequence from the naturally occurring promoter DNA sequence, or may be obtained through the use of PCR technology. See particularly, Mullis et al., Methods Enzymol. 155:335-350 (1987), and Higuchi, R. In PCR Technology: Principles and Applications for DNA Amplifications; Erlich, H. A., Ed.; Stockton Press Inc.: New York, 1989.

The terms “full complement” and “full-length complement” are used interchangeably herein, and refer to a complement of a given nucleotide sequence, wherein the complement and the nucleotide sequence consist of the same number of nucleotides and are 100% complementary.

The terms “substantially similar” and “corresponding substantially” as used herein refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences.

The isolated promoter sequence comprised in the recombinant DNA construct of the present disclosure can be modified to provide a range of constitutive expression levels of the heterologous nucleotide sequence. Thus, less than the entire promoter regions may be utilized and the ability to drive expression of the coding sequence retained. However, it is recognized that expression levels of the mRNA may be decreased with deletions of portions of the promoter sequences. Likewise, the tissue-independent, constitutive nature of expression may be changed.

Modifications of the isolated promoter sequences of the present disclosure can provide for a range of constitutive expression of the heterologous nucleotide sequence. Thus, they may be modified to be weak constitutive promoters or strong constitutive promoters. Generally, by “weak promoter” is intended a promoter that drives expression of a coding sequence at a low level. By “low level” is intended at levels about 1/10,000 transcripts to about 1/100,000 transcripts to about 1/500,000 transcripts. Conversely, a strong promoter drives expression of a coding sequence at high level, or at about 1/10 transcripts to about 1/100 transcripts to about 1/1,000 transcripts.

Moreover, the skilled artisan recognizes that substantially similar nucleic acid sequences encompassed by this disclosure are also defined by their ability to hybridize, under moderately stringent conditions (for example, 0.5×SSC, 0.1% SDS, 60° C.) with the sequences exemplified herein, or to any portion of the nucleotide sequences reported herein and which are functionally equivalent to the promoter of the disclosure. Estimates of such homology are provided by either DNA-DNA or DNA-RNA hybridization under conditions of stringency as is well understood by those skilled in the art (Hames and Higgins, Eds.; In Nucleic Acid Hybridisation; IRL Press: Oxford, U. K., 1985). Stringency conditions can be adjusted to screen for moderately similar fragments, such as homologous sequences from distantly related organisms, to highly similar fragments, such as genes that duplicate functional enzymes from closely related organisms. Post-hybridization washes partially determine stringency conditions. One set of conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. Another set of stringent conditions uses higher temperatures in which the washes are identical to those above except for the temperature of the final two 30 min washes in 0.2×SSC, 0.5% SDS was increased to 60° C. Another set of highly stringent conditions uses two final washes in 0.1×SSC, 0.1% SDS at 65° C.

Preferred substantially similar nucleic acid sequences encompassed by this disclosure are those sequences that are 80% identical to the nucleic acid fragments reported herein or which are 80% identical to any portion of the nucleotide sequences reported herein. More preferred are nucleic acid fragments which are 90% identical to the nucleic acid sequences reported herein, or which are 90% identical to any portion of the nucleotide sequences reported herein. Most preferred are nucleic acid fragments which are 95% identical to the nucleic acid sequences reported herein, or which are 95% identical to any portion of the nucleotide sequences reported herein. It is well understood by one skilled in the art that many levels of sequence identity are useful in identifying related polynucleotide sequences. Useful examples of percent identities are those listed above, or also preferred is any integer percentage from 71% to 100%, such as 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

A “substantially homologous sequence” refers to variants of the disclosed sequences such as those that result from site-directed mutagenesis, as well as synthetically derived sequences. A substantially homologous sequence of the present disclosure also refers to those fragments of a particular promoter nucleotide sequence disclosed herein that operate to promote the constitutive expression of an operably linked heterologous nucleic acid fragment. These promoter fragments will comprise at least about 20 contiguous nucleotides, preferably at least about 50 contiguous nucleotides, more preferably at least about 75 contiguous nucleotides, even more preferably at least about 100 contiguous nucleotides of the particular promoter nucleotide sequence disclosed herein. The nucleotides of such fragments will usually comprise the TATA recognition sequence of the particular promoter sequence. Such fragments may be obtained by use of restriction enzymes to cleave the naturally occurring promoter nucleotide sequences disclosed herein; by synthesizing a nucleotide sequence from the naturally occurring promoter DNA sequence; or may be obtained through the use of PCR technology. See particularly, Mullis et al., Methods Enzymol. 155:335-350 (1987), and Higuchi, R. In PCR Technology: Principles and Applications for DNA Amplifications; Erlich, H. A., Ed.; Stockton Press Inc.: New York, 1989. Again, variants of these promoter fragments, such as those resulting from site-directed mutagenesis, are encompassed by the compositions of the present disclosure.

“Codon degeneracy” refers to divergence in the genetic code permitting variation of the nucleotide sequence without affecting the amino acid sequence of an encoded polypeptide. Accordingly, the instant disclosure relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

In one embodiment the % sequence identity is determined over the entire length of the molecule (nucleotide or amino acid).

A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Altschul, S. F. et al., J. Mol. Biol. 215:403-410 (1993)) and Gapped Blast (Altschul, S. F. et al., Nucleic Acids Res. 25:3389-3402 (1997)). BLASTN refers to a BLAST program that compares a nucleotide query sequence against a nucleotide sequence database.

“Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in nature with its own regulatory sequences.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

“Chimeric gene” or “recombinant expression construct”, which are used interchangeably, includes any gene that is not a native gene, comprising regulatory and coding sequences that are not found together in nature. Accordingly, a chimeric gene may comprise regulatory sequences and coding sequences that are derived from different sources.

“Coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

An “intron” is an intervening sequence in a gene that is transcribed into RNA but is then excised in the process of generating the mature mRNA. The term is also used for the excised RNA sequences. An “exon” is a portion of the sequence of a gene that is transcribed and is found in the mature messenger RNA derived from the gene, but is not necessarily a part of the sequence that encodes the final gene product.

The “translation leader sequence” refers to a polynucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D., Molecular Biotechnology 3:225 (1995)).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., Plant Cell 1:671-680 (1989).

“RNA transcript” refers to a product resulting from RNA polymerase-catalyzed transcription of a DNA sequence. When an RNA transcript is a perfect complimentary copy of a DNA sequence, it is referred to as a primary transcript or it may be a RNA sequence derived from posttranscriptional processing of a primary transcript and is referred to as a mature RNA. “Messenger RNA” (“mRNA”) refers to RNA that is without introns and that can be translated into protein by the cell. “cDNA” refers to a DNA that is complementary to and synthesized from an mRNA template using the enzyme reverse transcriptase. The cDNA can be single-stranded or converted into the double-stranded by using the Klenow fragment of DNA polymerase I. “Sense” RNA refers to RNA transcript that includes mRNA and so can be translated into protein within a cell or in vitro. “Antisense RNA” refers to a RNA transcript that is complementary to all or part of a target primary transcript or mRNA and that blocks expression or transcripts accumulation of a target gene (U.S. Pat. No. 5,107,065). The complementarity of an antisense RNA may be with any part of the specific gene transcript, i.e. at the 5′ non-coding sequence, 3′ non-coding sequence, introns, or the coding sequence. “Functional RNA” refers to antisense RNA, ribozyme RNA, or other RNA that may not be translated but yet has an effect on cellular processes.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The terms “initiate transcription”, “initiate expression”, “drive transcription”, and “drive expression” are used interchangeably herein and all refer to the primary function of a promoter. As detailed throughout this disclosure, a promoter is a non-coding genomic DNA sequence, usually upstream (5′) to the relevant coding sequence, and its primary function is to act as a binding site for RNA polymerase and initiate transcription by the RNA polymerase. Additionally, there is “expression” of RNA, including functional RNA, or the expression of polypeptide for operably linked encoding nucleotide sequences, as the transcribed RNA ultimately is translated into the corresponding polypeptide.

The term “expression”, as used herein, refers to the production of a functional end-product e.g., an mRNA or a protein (precursor or mature).

The term “expression cassette” as used herein, refers to a discrete nucleic acid fragment into which a nucleic acid sequence or fragment can be moved.

Expression or overexpression of a gene involves transcription of the gene and translation of the mRNA into a precursor or mature protein. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression or transcript accumulation of identical or substantially similar foreign or endogenous genes (U.S. Pat. No. 5,231,020). The mechanism of co-suppression may be at the DNA level (such as DNA methylation), at the transcriptional level, or at posttranscriptional level.

Co-suppression constructs in plants previously have been designed by focusing on overexpression of a nucleic acid sequence having homology to an endogenous mRNA, in the sense orientation, which results in the reduction of all RNA having homology to the overexpressed sequence (see Vaucheret et al., Plant J. 16:651-659 (1998); and Gura, Nature 404:804-808 (2000)). The overall efficiency of this phenomenon is low, and the extent of the RNA reduction is widely variable. Recent work has described the use of “hairpin” structures that incorporate all, or part, of an mRNA encoding sequence in a complementary orientation that results in a potential “stem-loop” structure for the expressed RNA (PCT Publication No. WO 99/53050 published on Oct. 21, 1999; and PCT Publication No. WO 02/00904 published on Jan. 3, 2002). This increases the frequency of co-suppression in the recovered transgenic plants. Another variation describes the use of plant viral sequences to direct the suppression, or “silencing”, of proximal mRNA encoding sequences (PCT Publication No. WO 98/36083 published on Aug. 20, 1998). Genetic and molecular evidences have been obtained suggesting that dsRNA mediated mRNA cleavage may have been the conserved mechanism underlying these gene silencing phenomena (Elmayan et al., Plant Cell 10:1747-1757 (1998); Galun, In Vitro Cell. Dev. Biol. Plant 41(2):113-123 (2005); Pickford et al, Cell. Mol. Life Sci. 60(5):871-882 (2003)).

As stated herein, “suppression” refers to a reduction of the level of enzyme activity or protein functionality (e.g., a phenotype associated with a protein) detectable in a transgenic plant when compared to the level of enzyme activity or protein functionality detectable in a non-transgenic or wild type plant with the native enzyme or protein. The level of enzyme activity in a plant with the native enzyme is referred to herein as “wild type” activity. The level of protein functionality in a plant with the native protein is referred to herein as “wild type” functionality. The term “suppression” includes lower, reduce, decline, decrease, inhibit, eliminate and prevent. This reduction may be due to a decrease in translation of the native mRNA into an active enzyme or functional protein. It may also be due to the transcription of the native DNA into decreased amounts of mRNA and/or to rapid degradation of the native mRNA. The term “native enzyme” refers to an enzyme that is produced naturally in a non-transgenic or wild type cell. The terms “non-transgenic” and “wild type” are used interchangeably herein.

“Altering expression” refers to the production of gene product(s) in transgenic organisms in amounts or proportions that differ significantly from the amount of the gene product(s) produced by the corresponding wild-type organisms (i.e., expression is increased or decreased).

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation. Host organisms containing the transformed nucleic acid fragments are referred to as “transgenic” organisms.

“Transient transformation” refers to the introduction of a nucleic acid fragment into the nucleus, or DNA-containing organelle, of a host organism resulting in gene expression without genetically stable inheritance.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or

Transgenic includes any cell, cell line, callus, tissue, plant part or plant, the genome of which has been altered by the presence of a heterologous nucleic acid, such as a recombinant DNA construct.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

The terms “monocot” and “monocotyledonous plant” are used interchangeably herein. A monocot of the current disclosure includes the Gramineae.

The terms “dicot” and “dicotyledonous plant” are used interchangeably herein. A dicot of the current disclosure includes the following families: Brassicaceae, Leguminosae, and Solanaceae.

“Progeny” comprises any subsequent generation of a plant.

A transgenic plant includes, for example, a plant which comprises within its genome a heterologous polynucleotide introduced by a transformation step. The heterologous polynucleotide can be stably integrated within the genome such that the polynucleotide is passed on to successive generations. The heterologous polynucleotide may be integrated into the genome alone or as part of a recombinant DNA construct. A transgenic plant can also comprise more than one heterologous polynucleotide within its genome. Each heterologous polynucleotide may confer a different trait to the transgenic plant. A heterologous polynucleotide can include a sequence that originates from a foreign species, or, if from the same species, can be substantially modified from its native form. Transgenic can include any cell, cell line, callus, tissue, plant part or plant, the genotype of which has been altered by the presence of heterologous nucleic acid including those transgenics initially so altered as well as those created by sexual crosses or asexual propagation from the initial transgenic. The alterations of the genome (chromosomal or extra-chromosomal) by conventional plant breeding methods, by genome editing procedures that do not result in an insertion of a foreign polynucleotide, or by naturally occurring events such as random cross-fertilization, non-recombinant viral infection, non-recombinant bacterial transformation, non-recombinant transposition, or spontaneous mutation are not intended to be regarded as transgenic.

In certain embodiments of the disclosure, a fertile plant is a plant that produces viable male and female gametes and is self-fertile. Such a self-fertile plant can produce a progeny plant without the contribution from any other plant of a gamete and the genetic material contained therein. Other embodiments of the disclosure can involve the use of a plant that is not self-fertile because the plant does not produce male gametes, or female gametes, or both, that are viable or otherwise capable of fertilization. As used herein, a “male sterile plant” is a plant that does not produce male gametes that are viable or otherwise capable of fertilization. As used herein, a “female sterile plant” is a plant that does not produce female gametes that are viable or otherwise capable of fertilization. It is recognized that male-sterile and female-sterile plants can be female-fertile and male-fertile, respectively. It is further recognized that a male fertile (but female sterile) plant can produce viable progeny when crossed with a female fertile plant and that a female fertile (but male sterile) plant can produce viable progeny when crossed with a male fertile plant.

“Transient expression” refers to the temporary expression of often reporter genes such as β-glucuronidase (GUS), fluorescent protein genes ZS-GREEN1, ZS-YELLOW1 N1, AM-CYAN1, DS-RED in selected certain cell types of the host organism in which the transgenic gene is introduced temporally by a transformation method. The transformed materials of the host organism are subsequently discarded after the transient gene expression assay.

Standard recombinant DNA and molecular cloning techniques used herein are well known in the art and are described more fully in Sambrook, J. et al., In Molecular Cloning: A Laboratory Manual; 2^{nd} ed.; Cold Spring Harbor Laboratory Press: Cold Spring Harbor, N. Y., 1989 (hereinafter “Sambrook et al., 1989”) or Ausubel, F. M., Brent, R., Kingston, R. E., Moore, D. D., Seidman, J. G., Smith, J. A. and Struhl, K., Eds.; In Current Protocols in Molecular Biology; John Wiley and Sons: New York, 1990 (hereinafter “Ausubel et al., 1990”).

“PCR” or “Polymerase Chain Reaction” is a technique for the synthesis of large quantities of specific DNA segments, consisting of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps comprises a cycle.

The terms “plasmid”, “vector” and “cassette” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “recombinant DNA construct” or “recombinant expression construct” is used interchangeably and refers to a discrete polynucleotide into which a nucleic acid sequence or fragment can be moved. Preferably, it is a plasmid vector or a fragment thereof comprising the promoters of the present disclosure. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by PCR and Southern analysis of DNA, RT-PCR and Northern analysis of mRNA expression, Western analysis of protein expression, or phenotypic analysis.

Various changes in phenotype are of interest including, but not limited to, modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic characteristics and traits such as yield and heterosis increase, the choice of genes for transformation will change accordingly. General categories of genes of interest include, but are not limited to, those genes involved in information, such as zinc fingers, those involved in communication, such as kinases, and those involved in housekeeping, such as heat shock proteins. More specific categories of transgenes, for example, include, but are not limited to, genes encoding important traits for agronomics, insect resistance, disease resistance, herbicide resistance, sterility, grain or seed characteristics, and commercial products. Genes of interest include, generally, those involved in oil, starch, carbohydrate, or nutrient metabolism as well as those affecting seed size, plant development, plant growth regulation, and yield improvement. Plant development and growth regulation also refer to the development and growth regulation of various parts of a plant, such as the flower, seed, root, leaf and shoot.

Other commercially desirable traits are genes and proteins conferring cold, heat, salt, and drought resistance.

Disease and/or insect resistance genes may encode resistance to pests that have great yield drag such as for example, anthracnose, soybean mosaic virus, soybean cyst nematode, root-knot nematode, brown leaf spot, Downy mildew, purple seed stain, seed decay and seedling diseases caused commonly by the fungi—Pythium sp., Phytophthora sp., Rhizoctonia sp., Diaporthe sp. Bacterial blight caused by the bacterium Pseudomonas syringae pv. Glycinea. Genes conferring insect resistance include, for example, Bacillus thuringiensis toxic protein genes (U.S. Pat. Nos. 5,366,892; 5,747,450; 5,737,514; 5,723,756; 5,593,881; and Geiser et al (1986) Gene 48:109); lectins (Van Damme et al. (1994) Plant Mol. Biol. 24:825); and the like.

Herbicide resistance traits may include genes coding for resistance to herbicides that act to inhibit the action of acetolactate synthase (ALS), in particular the sulfonylurea-type herbicides (e.g., the acetolactate synthase ALS gene containing mutations leading to such resistance, in particular the S4 and/or HRA mutations). The ALS-gene mutants encode resistance to the herbicide chlorsulfuron. Glyphosate acetyl transferase (GAT) is an N-acetyltransferase from Bacillus licheniformis that was optimized by gene shuffling for acetylation of the broad spectrum herbicide, glyphosate, forming the basis of a novel mechanism of glyphosate tolerance in transgenic plants (Castle et al. (2004) Science 304, 1151-1154).

Antibiotic resistance genes include, for example, neomycin phosphotransferase (npt) and hygromycin phosphotransferase (hpt). Two neomycin phosphotransferase genes are used in selection of transformed organisms: the neomycin phosphotransferase I (nptI) gene and the neomycin phosphotransferase II (nptII) gene. The second one is more widely used. It was initially isolated from the transposon Tn5 that was present in the bacterium strain Escherichia coli K12. The gene codes for the aminoglycoside 3′-phosphotransferase (denoted aph(3′)-II or NPTII) enzyme, which inactivates by phosphorylation a range of aminoglycoside antibiotics such as kanamycin, neomycin, geneticin and paroromycin. NPTII is widely used as a selectable marker for plant transformation. It is also used in gene expression and regulation studies in different organisms in part because N-terminal fusions can be constructed that retain enzyme activity. NPTII protein activity can be detected by enzymatic assay. In other detection methods, the modified substrates, the phosphorylated antibiotics, are detected by thin-layer chromatography, dot-blot analysis or polyacrylamide gel electrophoresis. Plants such as maize, cotton, tobacco, Arabidopsis, flax, soybean and many others have been successfully transformed with the nptII gene.

The hygromycin phosphotransferase (denoted hpt, hph or aphIV) gene was originally derived from Escherichia coli. The gene codes for hygromycin phosphotransferase (HPT), which detoxifies the aminocyclitol antibiotic hygromycin B. A large number of plants have been transformed with the hpt gene and hygromycin B has proved very effective in the selection of a wide range of plants, including monocotyledonous. Most plants exhibit higher sensitivity to hygromycin B than to kanamycin, for instance cereals. Likewise, the hpt gene is used widely in selection of transformed mammalian cells. The sequence of the hpt gene has been modified for its use in plant transformation. Deletions and substitutions of amino acid residues close to the carboxy (C)-terminus of the enzyme have increased the level of resistance in certain plants, such as tobacco. At the same time, the hydrophilic C-terminus of the enzyme has been maintained and may be essential for the strong activity of HPT. HPT activity can be checked using an enzymatic assay. A non-destructive callus induction test can be used to verify hygromycin resistance.

Genes involved in plant growth and development have been identified in plants. One such gene, which is involved in cytokinin biosynthesis, is isopentenyl transferase (IPT). Cytokinin plays a critical role in plant growth and development by stimulating cell division and cell differentiation (Sun et al. (2003), Plant Physiol. 131: 167-176).

Calcium-dependent protein kinases (CDPK), a family of serine-threonine kinase found primarily in the plant kingdom, are likely to function as sensor molecules in calcium-mediated signaling pathways. Calcium ions are important second messengers during plant growth and development (Harper et al. Science 252, 951-954 (1993); Roberts et al. Curr. Opin. Cell Biol. 5, 242-246 (1993); Roberts et al. Annu. Rev. Plant Mol. Biol. 43, 375-414 (1992)).

Nematode responsive protein (NRP) is produced by soybean upon the infection of soybean cyst nematode. NRP has homology to a taste-modifying glycoprotein miraculin and the NF34 protein involved in tumor formation and hyper response induction. NRP is believed to function as a defense-inducer in response to nematode infection (Tenhaken et al. BMC Bioinformatics 6:169 (2005)).

The quality of seeds and grains is reflected in traits such as levels and types of fatty acids or oils, saturated and unsaturated, quality and quantity of essential amino acids, and levels of carbohydrates. Therefore, commercial traits can also be encoded on a gene or genes that could increase for example methionine and cysteine, two sulfur containing amino acids that are present in low amounts in soybeans. Cystathionine gamma synthase (CGS) and serine acetyl transferase (SAT) are proteins involved in the synthesis of methionine and cysteine, respectively.

Other commercial traits can encode genes to increase for example monounsaturated fatty acids, such as oleic acid, in oil seeds. Soybean oil for example contains high levels of polyunsaturated fatty acids and is more prone to oxidation than oils with higher levels of monounsaturated and saturated fatty acids. High oleic soybean seeds can be prepared by recombinant manipulation of the activity of oleoyl 12-desaturase (Fad2). High oleic soybean oil can be used in applications that require a high degree of oxidative stability, such as cooking for a long period of time at an elevated temperature.

Raffinose saccharides accumulate in significant quantities in the edible portion of many economically significant crop species, such as soybean (Glycine max L. Merrill), sugar beet (Beta vulgaris), cotton (Gossypium hirsutum L.), canola (Brassica sp.) and all of the major edible leguminous crops including beans (Phaseolus sp.), chick pea (Cicer arietinum), cowpea (Vigna unguiculata), mung bean (Vigna radiata), peas (Pisum sativum), lentil (Lens culinaris) and lupine (Lupinus sp.). Although abundant in many species, raffinose saccharides are an obstacle to the efficient utilization of some economically important crop species.

Down regulation of the expression of the enzymes involved in raffinose saccharide synthesis, such as galactinol synthase for example, would be a desirable trait.

In certain embodiments, the present disclosure contemplates the transformation of a recipient cell with more than one advantageous transgene. Two or more transgenes can be supplied in a single transformation event using either distinct transgene-encoding vectors, or a single vector incorporating two or more gene coding sequences. Any two or more transgenes of any description, such as those conferring herbicide, insect, disease (viral, bacterial, fungal, and nematode) or drought resistance, oil quantity and quality, or those increasing yield or nutritional quality may be employed as desired.

The transport of water through cell membranes is regulated in part by aquaporins or water channel proteins. These proteins are members of the larger family of major intrinsic proteins (MIPs) that are characterized by six transmembrane-spanning helices, cytosolic amino and carboxy termini, and a signature sequence (Maurel C., Annu. Rev. Plant Physiol. Plant Mol. Biol. 48:399-429 (1997); Agre et al., J. Biol. Chem. 273:14659-14662 (1998)). Aquaporins are classified in two main groups according to their sequence similarity with MIPs localized in the plasma membrane (plasma membrane intrinsic proteins or PIPs) or in the vacuolar membrane (tonoplast intrinsic proteins or TIPs). A great number of MIP homologs have been identified in plant species (Tyerman et al., J. Exp. Bot. 50:1055-1071 (1999); Schaffner A. R., Planta 204:131-139 (1998); Chaumont et al., Plant Physiol. 122:1025-1034 (2000)). In Arabidopsis, 23 expressed MIP genes were identified and classified into three groups: 11 plasma membrane intrinsic proteins, 11 tonoplast intrinsic proteins, and a single member that is most closely related to the Gm-NOD26 protein found in the bacteroid membranes of soybean nodules (Weig et al., Plant Physiol. 114: 1347-1357 (1997)). It is demonstrated herein that the soybean plasma intrinsic protein gene promoter GM-PIP1 can, in fact, be used as a constitutive promoter to drive expression of transgenes in plant, and that such promoter can be isolated and used by one skilled in the art.

This disclosure concerns an isolated nucleic acid fragment comprising a constitutive plasma membrane intrinsic protein gene promoter PIP1. This disclosure also concerns an isolated nucleic acid fragment comprising a promoter wherein said promoter consists essentially of the nucleotide sequence set forth in SEQ ID NO: 1, or an isolated polynucleotide comprising a promoter wherein said promoter comprises the nucleotide sequence set forth in SEQ ID NOs: 1, 2, 3, 4, 5, 6, 7 or 49 or a functional fragment of SEQ ID NOs: 1, 2, 3, 4, 5, 6, 7 or 49.

The expression patterns of PIP1 gene and its promoter are set forth in Examples 1-7.

The promoter activity of the soybean genomic DNA fragment SEQ ID NO:1 upstream of the PIP1 protein coding sequence was assessed by linking the fragment to a yellow fluorescence reporter gene, ZS-YELLOW1 N1 (YFP) (Tsien, Annu. Rev. Biochem. 67:509-544 (1998); Matz et al., Nat. Biotechnol. 17:969-973 (1999)), transforming the promoter:YFP expression cassette into soybean, and analyzing YFP expression in various cell types of the transgenic plants (see Example 6 and 7). YFP expression was detected in most parts of the transgenic plants. These results indicated that the nucleic acid fragment contained a constitutive promoter.

It is clear from the disclosure set forth herein that one of ordinary skill in the art could perform the following procedure:

1) operably linking the nucleic acid fragment containing the PIP1 promoter sequence to a suitable reporter gene; there are a variety of reporter genes that are well known to those skilled in the art, including the bacterial GUS gene, the firefly luciferase gene, and the cyan, green, red, and yellow fluorescent protein genes; any gene for which an easy and reliable assay is available can serve as the reporter gene.

2) transforming a chimeric PIP1 promoter:reporter gene expression cassette into an appropriate plant for expression of the promoter. There are a variety of appropriate plants which can be used as a host for transformation that are well known to those skilled in the art, including the dicots, Arabidopsis, tobacco, soybean, oilseed rape, peanut, sunflower, safflower, cotton, tomato, potato, cocoa and the monocots, corn, wheat, rice, barley and palm.

3) testing for expression of the PIP1 promoter in various cell types of transgenic plant tissues, e.g., leaves, roots, flowers, seeds, transformed with the chimeric PIP1 promoter:reporter gene expression cassette by assaying for expression of the reporter gene product.

In another aspect, this disclosure concerns a recombinant DNA construct comprising at least one heterologous nucleic acid fragment operably linked to any promoter, or combination of promoter elements, of the present disclosure. Recombinant DNA constructs can be constructed by operably linking the nucleic acid fragment of the disclosure PIP1 promoter or a fragment that is substantially similar and functionally equivalent to any portion of the nucleotide sequence set forth in SEQ ID NOs:1, 2, 3, 4, 5, 6, 7 or 49 to a heterologous nucleic acid fragment. Any heterologous nucleic acid fragment can be used to practice the disclosure. The selection will depend upon the desired application or phenotype to be achieved. The various nucleic acid sequences can be manipulated so as to provide for the nucleic acid sequences in the proper orientation. It is believed that various combinations of promoter elements as described herein may be useful in practicing the present disclosure.

In another aspect, this disclosure concerns a recombinant DNA construct comprising at least one acetolactate synthase (ALS) nucleic acid fragment operably linked to PIP1 promoter, or combination of promoter elements, of the present disclosure. The acetolactate synthase gene is involved in the biosynthesis of branched chain amino acids in plants and is the site of action of several herbicides including sulfonyl urea. Expression of a mutated acetolactate synthase gene encoding a protein that can no longer bind the herbicide will enable the transgenic plants to be resistant to the herbicide (U.S. Pat. No. 5,605,011, U.S. Pat. No. 5,378,824). The mutated acetolactate synthase gene is also widely used in plant transformation to select transgenic plants.

In another embodiment, this disclosure concerns host cells comprising either the recombinant DNA constructs of the disclosure as described herein or isolated polynucleotides of the disclosure as described herein. Examples of host cells which can be used to practice the disclosure include, but are not limited to, yeast, bacteria, and plants.

Plasmid vectors comprising the instant recombinant expression construct can be constructed. The choice of plasmid vector is dependent upon the method that will be used to transform host cells. The skilled artisan is well aware of the genetic elements that must be present on the plasmid vector in order to successfully transform, select and propagate host cells containing the chimeric gene.

Methods for transforming dicots, primarily by use of Agrobacterium tumefaciens, and obtaining transgenic plants have been published, among others, for cotton (U.S. Pat. No. 5,004,863, U.S. Pat. No. 5,159,135); soybean (U.S. Pat. No. 5,569,834, U.S. Pat. No. 5,416,011); Brassica (U.S. Pat. No. 5,463,174); peanut (Cheng et al., Plant Cell Rep. 15:653-657 (1996), McKently et al., Plant Cell Rep. 14:699-703 (1995)); papaya (Ling et al., Bio/technology 9:752-758 (1991)); and pea (Grant et al., Plant Cell Rep. 15:254-258 (1995)). For a review of other commonly used methods of plant transformation see Newell, C. A., Mol. Biotechnol. 16:53-65 (2000). One of these methods of transformation uses Agrobacterium rhizogenes (Tepfler, M. and Casse-Delbart, F., Microbiol. Sci. 4:24-28 (1987)). Transformation of soybeans using direct delivery of DNA has been published using PEG fusion (PCT Publication No. WO 92/17598), electroporation (Chowrira et al., Mol. Biotechnol. 3:17-23 (1995); Christou et al., Proc. Natl. Acad. Sci. U.S.A. 84:3962-3966 (1987)), microinjection, or particle bombardment (McCabe et al., Biotechnology 6:923-926 (1988); Christou et al., Plant Physiol. 87:671-674 (1988)).

There are a variety of methods for the regeneration of plants from plant tissues. The particular method of regeneration will depend on the starting plant tissue and the particular plant species to be regenerated. The regeneration, development and cultivation of plants from single plant protoplast transformants or from various transformed explants is well known in the art (Weissbach and Weissbach, Eds.; In Methods for Plant Molecular Biology; Academic Press, Inc.: San Diego, Calif., 1988). This regeneration and growth process typically includes the steps of selection of transformed cells, culturing those individualized cells through the usual stages of embryonic development or through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

In addition to the above discussed procedures, practitioners are familiar with the standard resource materials which describe specific conditions and procedures for the construction, manipulation and isolation of macromolecules (e.g., DNA molecules, plasmids, etc.), generation of recombinant DNA fragments and recombinant expression constructs and the screening and isolating of clones, (see for example, Sambrook, J. et al., In Molecular Cloning: A Laboratory Manual; 2^{nd} ed.; Cold Spring Harbor Laboratory Press: Cold Spring Harbor, N. Y., 1989; Maliga et al., In Methods in Plant Molecular Biology; Cold Spring Harbor Press, 1995; Birren et al., In Genome Analysis: Detecting Genes, 1; Cold Spring Harbor: New York, 1998; Birren et al., In Genome Analysis: Analyzing DNA, 2; Cold Spring Harbor: New York, 1998; Clark, Ed., In Plant Molecular Biology: A Laboratory Manual; Springer: New York, 1997).

The skilled artisan will also recognize that different independent transformation events will result in different levels and patterns of expression of the chimeric genes (Jones et al., EMBO J. 4:2411-2418 (1985); De Almeida et al., Mol. Gen. Genetics 218:78-86 (1989)). Thus, multiple events must be screened in order to obtain lines displaying the desired expression level and pattern. Such screening may be accomplished by Northern analysis of mRNA expression, Western analysis of protein expression, or phenotypic analysis. Also of interest are seeds obtained from transformed plants displaying the desired gene expression profile.

The level of activity of the PIP1 promoter is weaker than that of many known strong promoters, such as the CaMV 35S promoter (Atanassova et al., Plant Mol. Biol. 37:275-285 (1998); Battraw and Hall, Plant Mol. Biol. 15:527-538 (1990); Holtorf et al., Plant Mol. Biol. 29:637-646 (1995); Jefferson et al., EMBO J. 6:3901-3907 (1987); Wilmink et al., Plant Mol. Biol. 28:949-955 (1995)), the Arabidopsis oleosin promoters (Plant et al., Plant Mol. Biol. 25:193-205 (1994); Li, Texas A&M University Ph.D. dissertation, pp. 107-128 (1997)), the Arabidopsis ubiquitin extension protein promoters (Callis et al., J. Biol. Chem. 265(21):12486-12493 (1990)), a tomato ubiquitin gene promoter (Rollfinke et al., Gene 211:267-276 (1998)), a soybean heat shock protein promoter, and a maize H3 histone gene promoter (Atanassova et al., Plant Mol. Biol. 37:275-285 (1998)). Universal weak expression of chimeric genes in most plant cells makes the PIP1 promoter of the instant disclosure especially useful when moderate constitutive expression of a target heterologous nucleic acid fragment is required.

Another general application of the PIP1 promoter of the disclosure is to construct chimeric genes that can be used to reduce expression of at least one heterologous nucleic acid fragment in a plant cell. To accomplish this, a chimeric gene designed for gene silencing of a heterologous nucleic acid fragment can be constructed by linking the fragment to the PIP1 promoter of the present disclosure. (See U.S. Pat. No. 5,231,020, and PCT Publication No. WO 99/53050 published on Oct. 21, 1999, PCT Publication No. WO 02/00904 published on Jan. 3, 2002, and PCT Publication No. WO 98/36083 published on Aug. 20, 1998, for methodology to block plant gene expression via cosuppression.) Alternatively, a chimeric gene designed to express antisense RNA for a heterologous nucleic acid fragment can be constructed by linking the fragment in reverse orientation to the PIP1 promoter of the present disclosure. (See U.S. Pat. No. 5,107,065 for methodology to block plant gene expression via antisense RNA.) Either the cosuppression or antisense chimeric gene can be introduced into plants via transformation. Transformants wherein expression of the heterologous nucleic acid fragment is decreased or eliminated are then selected.

This disclosure also concerns a method of altering (increasing or decreasing) the expression of at least one heterologous nucleic acid fragment in a plant cell which comprises:
(a) transforming a plant cell with the recombinant expression construct described herein;
(b) growing fertile mature plants from the transformed plant cell of step (a);
(c) selecting plants containing a transformed plant cell wherein the expression of the heterologous nucleic acid fragment is increased or decreased.

Transformation and selection can be accomplished using methods well-known to those skilled in the art including, but not limited to, the methods described herein.

Non-limiting examples of methods and compositions disclosed herein are as follows:
1. A recombinant DNA construct comprising:
   (a) a nucleotide sequence comprising the sequence set forth in SEQ ID NO:1, SEQ ID NO:2, SEQ ID NO:3, SEQ ID NO:4, SEQ ID NO:5, or SEQ ID NO:6, or SEQ ID NO: 49, or a functional fragment thereof; or,
   (b) a full-length complement of (a); or,
   (c) a nucleotide sequence comprising a sequence having at least 71° A sequence identity, based on the Clustal V method of alignment with pairwise alignment default parameters (KTUPLE=2, GAP PENALTY=5, WINDOW=4 and DIAGONALS SAVED=4), when compared to the nucleotide sequence of (a);
   wherein said nucleotide sequence is a promoter.
2. The recombinant DNA construct of embodiment 1, wherein the promoter is a constitutive promoter.
3. The recombinant DNA construct of embodiment 1, wherein said nucleotide sequence has at least 95% identity, based on the Clustal V method of alignment with pairwise alignment default parameters (KTUPLE=2, GAP PENALTY=5, WINDOW=4 and DIAGONALS SAVED=4), when compared to any one of the sequence set forth in SEQ ID NO:1, SEQ ID NO:2, SEQ ID NO:3, SEQ ID NO:4, SEQ ID NO:5, SEQ ID NO:6, or SEQ ID NO:49.
4. The recombinant DNA construct of embodiment 3, wherein said nucleotide sequence is SEQ ID NO: 49.
5. A vector comprising the recombinant DNA construct of embodiment 1.
6. A cell comprising the recombinant DNA construct of embodiment 1.
7. The cell of embodiment 6, wherein the cell is a plant cell.
8. A transgenic plant having stably incorporated into its genome the recombinant DNA construct of embodiment 1.
9. The transgenic plant of embodiment 8 wherein said plant is a dicot plant.
10. The transgenic plant of embodiment 8 wherein the plant is soybean.
11. A transgenic seed produced by the transgenic plant of embodiment 8.
12. The recombinant DNA construct according to embodiment 1, wherein the at least one heterologous nucleotide sequence codes for a gene selected from the group consisting of: a reporter gene, a selection marker, a disease resistance conferring gene, a herbicide resistance conferring gene, an insect resistance conferring gene; a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in cold resistance, a gene involved in heat resistance and a gene involved in salt resistance in plants.
13. The recombinant DNA construct according to embodiment 1, wherein the at least one heterologous nucleotide sequence encodes a protein selected from the group consisting of: a reporter protein, a selection marker, a protein conferring disease resistance, protein conferring herbicide resistance, protein conferring insect resistance; protein involved in carbohydrate metabolism, protein involved in fatty acid metabolism, protein involved in amino acid metabolism, protein involved in plant development, protein involved in plant growth regulation, protein involved in yield improvement, protein involved in drought resistance, protein involved in cold resistance, protein involved in heat resistance and protein involved in salt resistance in plants.
14. A method of expressing a coding sequence or a functional RNA in a plant comprising:
   a) introducing the recombinant DNA construct of embodiment 1 into the plant, wherein the at least one heterologous nucleotide sequence comprises a coding sequence or a functional RNA;
   b) growing the plant of step a); and
   c) selecting a plant displaying expression of the coding sequence or the functional RNA of the recombinant DNA construct.
15. A method of transgenically altering a marketable plant trait, comprising:
   a) introducing a recombinant DNA construct of embodiment 1 into the plant;
   b) growing a fertile, mature plant resulting from step a); and
   c) selecting a plant expressing the at least one heterologous nucleotide sequence in at least one plant tissue based on the altered marketable trait.
16. The method of embodiment 15 wherein the marketable trait is selected from the group consisting of: disease resistance, herbicide resistance, insect resistance carbohydrate metabolism, fatty acid metabolism, amino acid metabolism, plant development, plant growth regulation, yield improvement, drought resistance, cold resistance, heat resistance, and salt resistance.
17. A method for altering expression of at least one heterologous nucleic acid fragment in plant comprising:
   (a) transforming a plant cell with the recombinant DNA construct of embodiment 1;
   (b) growing fertile mature plants from transformed plant cell of step (a); and
   (c) selecting plants containing the transformed plant cell wherein the expression of the heterologous nucleic acid fragment is increased or decreased.
18. The method of embodiment 17 wherein the plant is a soybean plant.
19. A method for expressing a yellow fluorescent protein ZS-YELLOW1 N1 in a host cell comprising:
   (a) transforming a host cell with the recombinant DNA construct of embodiment 1; and,
   (b) growing the transformed host cell under conditions that are suitable for expression of the recombinant DNA construct, wherein expression of the recombinant DNA construct results in production of increased levels of ZS-YELLOW1 N1 protein in the transformed host cell when compared to a corresponding non-transformed host cell.
20. A plant stably transformed with a recombinant DNA construct comprising a soybean constitutive promoter and a heterologous nucleic acid fragment operably linked to said constitutive promoter, wherein said constitutive promoter is a capable of controlling expression of said heterologous nucleic acid fragment in a plant cell, and further wherein said constitutive promoter comprises any of the sequences set forth in SEQ ID NO:1, SEQ ID NO:2, SEQ ID NO:3, SEQ ID NO:4, SEQ ID NO:5, SEQ ID NO:6, or SEQ ID NO:49.

### EXAMPLES

The present disclosure is further defined in the following Examples, in which parts and percentages are by weight and degrees are Celsius, unless otherwise stated. Sequences of promoters, cDNA, adaptors, and primers listed in this disclosure all are in the 5′ to 3′ orientation unless described otherwise. Techniques in molecular biology were typically performed as described in Ausubel, F. M. et al., In Current Protocols in Molecular Biology; John Wiley and Sons: New York, 1990 or Sambrook, J. et al., In Molecular Cloning: A Laboratory Manual; 2^{nd} ed.; Cold Spring Harbor Laboratory Press: Cold Spring Harbor, N. Y., 1989 (hereinafter “Sambrook et al., 1989”). It should be understood that these Examples, while indicating preferred embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended embodiments.

The disclosure of each reference set forth herein is incorporated herein by reference in its entirety.

### Example 1

### Identification of Soybean Constitutive Promoter Candidate Genes

Soybean expression sequence tags (EST) were generated by sequencing randomly selected clones from cDNA libraries constructed from different soybean tissues. Multiple EST sequences could often be found with different lengths representing the different regions of the same soybean gene. If more EST sequences representing the same gene are frequently found from a tissue-specific cDNA library such as a flower library than from a leaf library, there is a possibility that the represented gene could be a flower preferred gene candidate. Likewise, if similar numbers of ESTs for the same gene were found in various libraries constructed from different tissues, the represented gene could be a constitutively expressed gene. Multiple EST sequences representing the same soybean gene were compiled electronically based on their overlapping sequence homology into a unique full length sequence representing the gene. These assembled unique gene sequences were accumulatively collected in Pioneer Hi-Bred Intl proprietary searchable databases.

To identify constitutive promoter candidate genes, searches were performed to look for gene sequences that were found at similar frequencies in leaf, root, flower, embryos, pod, and also in other tissues. One unique gene PSO332986 was identified in the search to be a moderate constitutive gene candidate. PSO332986 cDNA sequence (SEQ ID NO:17) as well as its putative translated protein sequence (SEQ ID NO:18) were used to search National Center for Biotechnology Information (NCBI) databases. Both PSO332986 nucleotide and amino acid sequences were found to have high homology to plasma membrane intrinsic protein (aquaporin) genes discovered in several plant species including an identical soybean cDNA (SEQ ID NO:48; NCBI accession AK246127.1; Umezawa et al., DNA Res. 15:333-346 (2008)).

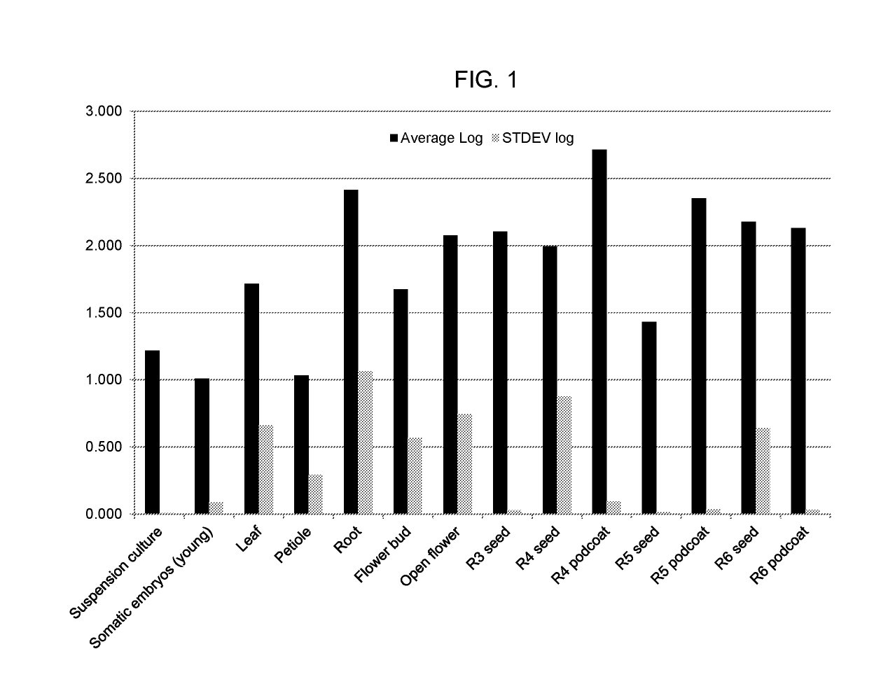

The expression profile of PSO332986 was confirmed and extended by analyzing 14 different soybean tissues using the relative quantitative RT-PCR technique with a ABI7500 real time PCR system (Applied Biosystems, Foster City, Calif.). Fourteen soybean tissues, somatic embryo, somatic embryo one week on charcoal plate, leaf, leaf petiole, root, flower bud, open flower, R3 pod, R4 seed, R4 pod coat, R5 seed, R5 pod coat, R6 seed, R6 pod coat were collected from cultivar ‘Jack’ and flash frozen in liquid nitrogen. The seed and pod development stages were defined according to descriptions in Fehr and Caviness, IWSRBC 80:1-12 (1977). Total RNA was extracted with TRIzol® reagents (Invitrogen, Carlsbad, Calif.) and treated with DNase I to remove any trace amount of genomic DNA contamination. The first strand cDNA was synthesized using the Superscript™ III reverse transcriptase (Invitrogen). Regular PCR analysis was done to confirm that the cDNA was free of any genomic DNA using primers shown in SEQ ID NO:25 and 26. The primers are specific to the 5′UTR intron/exon junction regions of a soybean S-adenosylmethionine synthetase gene promoter SAMS (U.S. Pat. No. 7,217,858). PCR using this primer set will amplify a 967 bp DNA fragment from any soybean genomic DNA template and a 376 bp DNA fragment from the cDNA template. Genome DNA-free cDNA aliquots were used in quantitative RT-PCR analysis in which an endogenous soybean ATP sulfurylase gene (ATPS) was used as an internal control and wild type soybean genomic DNA was used as the calibrator for relative quantification. PSO332986 gene-specific primers SEQ ID NO:27 and 28 and ATPS gene-specific primers SEQ ID NO:29 and 30 were used in separate PCR reactions using the Power Sybr® Green real time PCR master mix (Applied Biosystems). PCR reaction data were captured and analyzed using the sequence detection software provided with the ABI7500 real time PCR system. The logarithm values of relative quantifications of gene expression in the fourteen tissues were graphed for comparison. The qRT-PCR expression profiling of the PSO332986 PIP1 gene confirmed its moderate constitutive expression in all checked tissues (FIG. 1).

Solexa digital gene expression dual-tag-based mRNA profiling using the Illumina (Genome Analyzer) GA2 machine is a restriction enzyme site anchored tag-based technology, in this regard similar to Mass Parallel Signature Sequence transcript profiling technique (MPSS), but with two key differences (Morrissy et al., Genome Res. 19:1825-1835 (2009); Brenner et al., Proc. Natl. Acad. Sci. USA 97:1665-70 (2000)). Firstly, not one but two restriction enzymes were used, DpnII and NlaI, the combination of which increases gene representation and helps moderate expression variances. The aggregate occurrences of all the resulting sequence reads emanating from these DpnII and NlaI sites, with some repetitive tags removed computationally, were used to determine the overall gene expression levels. Secondly, the tag read length used here is 21 nucleotides, giving the Solexa tag data higher gene match fidelity than the shorter 17-mers used in MPSS. Soybean mRNA global gene expression profiles are stored in a Pioneer proprietary database TDExpress (Tissue Development Expression Browser). Candidate genes with different expression patterns can be searched, retrieved, and further evaluated.

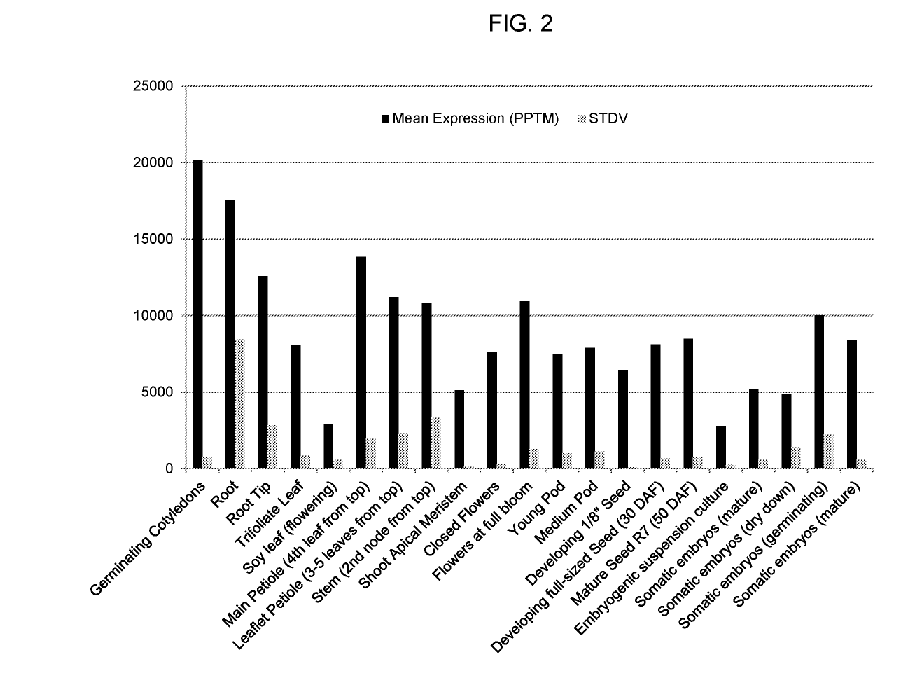

The plasma membrane intrinsic protein gene PSO332986 (PIP1) corresponds to predicted gene Glyma14g06680.1 in the soybean genome, sequenced by the DOE-JGI Community Sequencing Program consortium (Schmutz J. et al., Nature 463:178-183, 2010). The PIP1 expression profiles in twenty tissues were retrieved from the TDExpress database using the gene ID Glyma14g06680.1 and presented as parts per ten millions (PP™) averages of three experimental repeats (FIG. 2). The PIP1 gene is expressed in all checked tissues at moderate levels with the highest expression detected in germinating cotyledons, which is consistent with its EST profiles as a moderately expressed constitutive gene.

### Example 2

### Isolation of Soybean PIP1 Promoter

A BAC clone SBH172F4 corresponding to PSO332986 was identified from the screening of Pioneer Hi-Bred Intl propriety soybean BAC libraries using PSO332986 gene-specific primers SEQ ID NO:31 and 32 by PCR (polymerase chain reaction). The BAC clone was partially sequenced to reveal an approximately 2 Kb sequence upstream of PSO332986 PIP1 gene coding region. The primers shown in SEQ ID NO:8 and 9 were then designed to amplify the putative full length 1592 bp PIP1 promoter from the BAC clone DNA by PCR. SEQ ID NO:8 contains a recognition site for the restriction enzyme XmaI. SEQ ID NO:9 contains a recognition site for the restriction enzyme NcoI. The PIP1 promoter was later cloned into an expression vector using the restriction enzymes sites to study its functions.

PCR cycle conditions were 94° C. for 4 minutes; 35 cycles of 94° C. for 30 seconds, 60° C. for 1 minute, and 68° C. for 2 minutes; and a final 68° C. for 5 minutes before holding at 4° C. using the Platinum high fidelity Taq DNA polymerase (Invitrogen). The PCR reaction was resolved using agarose gel electrophoresis to identify the right size PCR product representing the ˜1.6 Kb PIP1 promoter. The PCR fragment was first cloned into pCR2.1-TOPO vector by TA cloning (Invitrogen). Several clones containing the ˜1.6 Kb DNA insert were sequenced and confirmed to contain the same PIP1 promoter sequence as previously sequenced from the BAC clone SBH172F4. One clone with the correct PIP1 promoter sequence was selected and its plasmid DNA digested with XmaI and NcoI restriction enzymes to move the PIP1 promoter upstream of the ZS-YELLOW N1 (YFP) fluorescent reporter gene in QC386 (SEQ ID NO:19). Construct QC386 contains the recombination sites AttL1 and AttL2 (SEQ ID NO:42 and 43) to qualify as a GATEWAY® cloning entry vector (Invitrogen). The 1592 bp sequence upstream of the PIP1 gene PSO332986 start codon ATG including the XmaI and NcoI sites is herein designated as soybean PIP1 promoter of SEQ ID NO:1.

Comparison of SEQ ID NO:1 to a soybean cDNA library revealed that SEQ ID NO: 1 comprised a 5′ untranslated region (UTR) at its 3′ end of at least 65 base pairs (SEQ ID NO:50). It is known to one of skilled in the art that a 5′ UTR region can be altered (deletion or substitutions of bases) or replaced by an alternative 5′UTR while maintaining promoter activity.

### Example 3

### PIP1 Promoter Copy Number Analysis

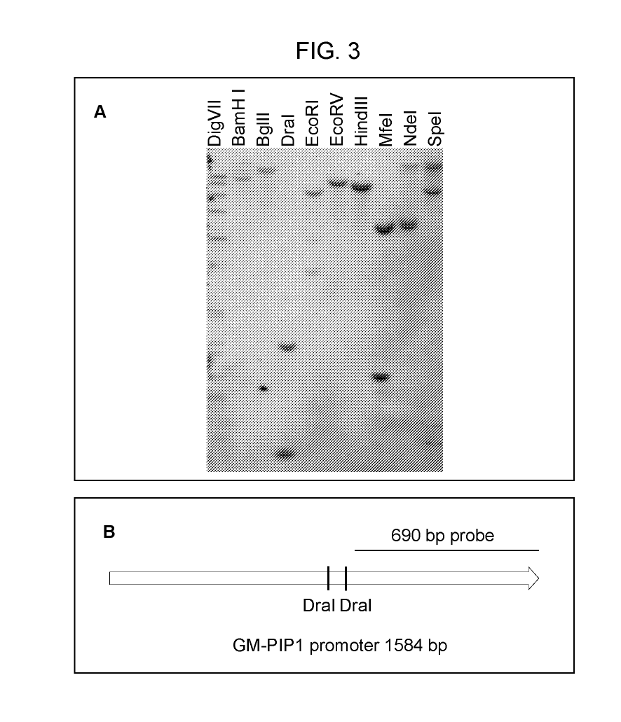

Southern hybridization analysis was performed to examine whether additional copies or sequences with significant similarity to the PIP1 promoter exist in the soybean genome. Soybean ‘Jack’ wild type genomic DNA was digested with nine different restriction enzymes, BamHI, BgIII, DraI, EcoRI, EcoRV, HindIII, MfeI, NdeI, and SpeI and distributed in a 0.7% agarose gel by electrophoresis (FIG. 3A). The DNA was blotted onto Nylon membrane and hybridized at 60° C. with digoxigenin labeled PIP1 promoter DNA probe in Easy-Hyb Southern hybridization solution, and then sequentially washed 10 minutes with 2×SSC/0.1% SDS at room temperature and 3×10 minutes at 65° C. with 0.1×SSC/0.1% SDS according to the protocol provided by the manufacturer (Roche Applied Science, Indianapolis, Ind.). The PIP1 promoter probe was labeled by PCR using the DIG DNA labeling kit (Roche Applied Science) with primers PSO332986S2 (SEQ ID NO:14) and QC386-A (SEQ ID NO:10) and QC386 plasmid DNA (SEQ ID NO:19) as the template to make a 690 bp long probe covering the 3′ half of the PIP1 promoter (FIG. 3B).

Only DraI of the nine restriction enzymes would cut the 1584 bp PIP1 promoter sequence (SEQ ID NO:2), which has the artificially added XmaI and NcoI sites at the 5′ and 3′ ends of the PIP1 promoter removed, twice and all in the middle so only the 3′ PIP1 promoter fragment can be detected by Southern hybridization with the 690 bp PIP1 probe. None of the other eight restriction enzymes BamHI, BgIII, EcoRI, EcoRV, HindIII, MfeI, NdeI, and SpeI would cut the promoter. Therefore, only one band would be expected to be hybridized for each of the nine digestions if only one copy of PIP1 promoter sequence exists in soybean genome (FIG. 3B). The observation that one major band and one or two minor bands detected in most digestions suggests that, in addition to the PIP1 promoter sequence (SEQ ID NO:1), there is another sequence similar enough to be hybridized by the same 690 bp PIP1 probe in soybean genome (FIG. 3A). The DIGVII molecular markers used on the Southern blot are 8576, 7427, 6106, 4899, 3639, 2799, 1953, 1882, 1515, 1482, 1164, 992, 718, 710 bp.

Since the whole soybean genome sequence is now publically available (Schmutz J., et al., Nature 463:178-183, 2010), the PIP1 promoter copy numbers can also be evaluated by searching the soybean genome with the 1584 bp promoter sequence (SEQ ID NO:2). Consistent with above Southern analysis, only one identical sequence Gm14:4892693-4894273 matching the PIP1 promoter sequence 2-1583 bp is identified. The first and last base pairs of the 1584 bp PIP1 promoter do not match the genomic Gm14 sequence since they are also parts of artificially added XmaI and NcoI sites. The near full length PIP1 promoter sequence (15-1583 bp) also matches complementarily to sequence Gm02:47299218-47297625 significantly but with many small gaps. The region corresponding to the 690 bp PIP1 probe sequence contains long enough stretches of identical sequences to be hybridized by the Southern probe. This similar sequence may correspond to the often minor Southern bands (FIG. 3A).

A nucleotide sequence alignment of SEQ ID NO: 1, comprising the full length PIP1 promoter of the disclosure, and SEQ ID NO: 49, comprising a 1591 bp native soybean genomic DNA from Gm14:4892283 . . . 4893874 (Schmutz J. et al., Nature 463:178-183, 2010) revealed that SEQ ID NO:1 is 99.7% identical to SEQ ID NO:49, based on the Clustal V method of alignment with pairwise alignment default parameters (KTUPLE=2, GAP PENALTY=5, WINDOW=4 and DIAGONALS SAVED=4). Based on the data described in Examples 1-7, it is believed that SEQ ID NO:49 has promoter activity.

### Example 4

### PIP1:YFP Reporter Gene Constructs and Soybean Transformation

The PIP1:YFP cassette in GATEWAY® entry construct QC386 described in EXAMPLE 2 was moved into a GATEWAY® destination vector QC324i (SEQ ID NO:20) by LR Clonase® (Invitrogen) mediated DNA recombination between the attL1 and attL2 recombination sites (SEQ ID NO:42, and 43, respectively) in QC386 and the attR1-attR2 recombination sites (SEQ ID NO:44, and 45, respectively) in QC324i to make the final transformation construct QC389 (SEQ ID NO:21).

Since the destination vector QC324i already contains a soybean transformation selectable marker gene SAMS:ALS, the resulting DNA construct QC389 has the PIP1:YFP gene expression cassette linked to the SAMS:ALS cassette. Two 21 bp recombination sites attB1 and attB2 (SEQ ID NO:46, and 47, respectively) were newly created recombination sites resulting from DNA recombination between attL1 and attR1, and between attL2 and attR2, respectively. The 6880 bp DNA fragment containing the linked PIP1:YFP and SAMS:ALS expression cassettes was isolated from plasmid QC389 (SEQ ID NO:21) with Ascl digestion, separated from the vector backbone fragment by agarose gel electrophoresis, and purified from the gel with a DNA gel extraction kit (QIAGEN®, Valencia, Calif.). The purified DNA fragment was transformed to soybean cultivar Jack by the method of particle gun bombardment (Klein et al., Nature 327:70-73 (1987); U.S. Pat. No. 4,945,050) as described in detail below to study the PIP1 promoter activity in stably transformed soybean plants.

The same methodology as outlined above for the PIP1:YFP expression cassette construction and transformation can be used with other heterologous nucleic acid sequences encoding for example a reporter protein, a selection marker, a protein conferring disease resistance, protein conferring herbicide resistance, protein conferring insect resistance; protein involved in carbohydrate metabolism, protein involved in fatty acid metabolism, protein involved in amino acid metabolism, protein involved in plant development, protein involved in plant growth regulation, protein involved in yield improvement, protein involved in drought resistance, protein involved in cold resistance, protein involved in heat resistance and salt resistance in plants.

Soybean somatic embryos from the Jack cultivar were induced as follows. Cotyledons (˜3 mm in length) were dissected from surface sterilized, immature seeds and were cultured for 6-10 weeks in the light at 26° C. on a Murashige and Skoog media containing 0.7% agar and supplemented with 10 mg/ml 2,4-D (2,4-Dichlorophenoxyacetic acid). Globular stage somatic embryos, which produced secondary embryos, were then excised and placed into flasks containing liquid MS medium supplemented with 2,4-D (10 mg/ml) and cultured in the light on a rotary shaker. After repeated selection for clusters of somatic embryos that multiplied as early, globular staged embryos, the soybean embryogenic suspension cultures were maintained in 35 ml liquid media on a rotary shaker, 150 rpm, at 26° C. with fluorescent lights on a 16:8 hour day/night schedule. Cultures were subcultured every two weeks by inoculating approximately 35 mg of tissue into 35 ml of the same fresh liquid MS medium.

Soybean embryogenic suspension cultures were then transformed by the method of particle gun bombardment using a DuPont Biolistic™ PDS1000/HE instrument (Bio-Rad Laboratories, Hercules, Calif.). To 50 μl of a 60 mg/ml 1.0 mm gold particle suspension were added (in order): 30 μl of 30 ng/μl QC589 DNA fragment PIP1:YFP+SAMS:ALS, 20 μl of 0.1 M spermidine, and 25 μl of 5 M CaCl₂. The particle preparation was then agitated for 3 minutes, spun in a centrifuge for 10 seconds and the supernatant removed. The DNA-coated particles were then washed once in 400 μl 100% ethanol and resuspended in 45 μl of 100% ethanol. The DNA/particle suspension was sonicated three times for one second each. 5 μl of the DNA-coated gold particles was then loaded on each macro carrier disk.

Approximately 300-400 mg of a two-week-old suspension culture was placed in an empty 60×15 mm Petri dish and the residual liquid removed from the tissue with a pipette. For each transformation experiment, approximately 5 to 10 plates of tissue were bombarded. Membrane rupture pressure was set at 1100 psi and the chamber was evacuated to a vacuum of 28 inches mercury. The tissue was placed approximately 3.5 inches away from the retaining screen and bombarded once. Following bombardment, the tissue was divided in half and placed back into liquid media and cultured as described above.

Five to seven days post bombardment, the liquid media was exchanged with fresh media containing 30 μg/ml hygromycin B as selection agent. This selective media was refreshed weekly. Seven to eight weeks post bombardment, green, transformed tissue was observed growing from untransformed, necrotic embryogenic clusters. Isolated green tissue was removed and inoculated into individual flasks to generate new, clonally propagated, transformed embryogenic suspension cultures. Each clonally propagated culture was treated as an independent transformation event and subcultured in the same liquid MS media supplemented with 2,4-D (10 mg/ml) and 100 ng/ml chlorsulfuron selection agent to increase mass. The embryogenic suspension cultures were then transferred to agar solid MS media plates without 2,4-D supplement to allow somatic embryos to develop. A sample of each event was collected at this stage for quantitative PCR analysis.

Cotyledon stage somatic embryos were dried-down (by transferring them into an empty small Petri dish that was seated on top of a 10 cm Petri dish containing some agar gel to allow slow dry down) to mimic the last stages of soybean seed development. Dried-down embryos were placed on germination solid media and transgenic soybean plantlets were regenerated. The transgenic plants were then transferred to soil and maintained in growth chambers for seed production.

Genomic DNA were extracted from somatic embryo samples and analyzed by quantitative PCR using the 7500 real time PCR system (Applied Biosystems) with gene-specific primers and FAM-labeled fluorescence probes to check copy numbers of both the SAMS:ALS expression cassette and the PIP1:YFP expression cassette. The qPCR analysis was done in duplex reactions with a heat shock protein (HSP) gene as the endogenous controls and a transgenic DNA sample with a known single copy of SAMS:ALS or YFP transgene as the calibrator using the relative quantification methodology (Applied Biosystems). The endogenous control HSP probe was labeled with VIC and the target gene SAMS:ALS or YFP probe was labeled with FAM for the simultaneous detection of both fluorescent probes (Applied Biosystems).

The primers and probes used in the qPCR analysis are listed below.
SAMS forward primer: SEQ ID N0:33
FAM labeled ALS probe: SEQ ID N0:34
ALS reverse primer: SEQ ID N0:35
YFP forward primer: SEQ ID N0:36
FAM labeled YFP probe: SEQ ID N0:37
YFP reverse primer: SEQ ID N0:38
HSP forward primer: SEQ ID N0:39
VIC labeled HSP probe: SEQ ID N0:40
HSP reverse primer: SEQ ID N0:41

Only transgenic soybean events containing 1 or 2 copies of both the SAMS:ALS expression cassette and the PIP1:YFP expression cassette were selected for further gene expression evaluation and seed production (see Table 1). Events negative for YFP qPCR or with more than 2 copies for the SAMS:ALS qPCR were not further followed. YFP expressions are described in detail in EXAMPLE 7 and are also summarized in Table 1.

TABLE 1
Relative transgene copy numbers and YFP
expression of PIP1:YFP transgenic plants

| | YFP | YFP | SAMS:ALS |
|---|---|---|---|
| Event ID | Expression | qPCR | qPCR |
| 5469.1.1 | + | 0.4 | 0.5 |
| 5469.1.2 | + | 0.8 | 0.8 |
| 5469.3.1 | + | 1.0 | 1.3 |
| 5469.3.2 | + | 0.9 | 1.0 |
| 5469.3.5 | + | 0.7 | 0.9 |
| 5469.3.6 | + | 0.9 | 0.6 |
| 5469.3.7 | + | 0.9 | 1.3 |
| 5469.4.2 | + | 0.8 | 0.9 |
| 5469.4.3 | + | 2.1 | 2.2 |
| 5469.4.4 | + | 1.0 | 1.1 |
| 5469.5.2 | + | 1.1 | 1.1 |
| 5469.5.5 | + | 1.8 | 2.2 |
| 5469.5.7 | + | 1.1 | 0.6 |
| 5469.5.9 | + | 1.2 | 1.7 |
| 5469.7.1 | + | 0.9 | 1.0 |
| 5469.8.1 | + | 1.0 | 0.8 |

### Example 5

### Construction of PIP1 Promoter Deletion Constructs

To define the transcriptional elements controlling the PIP1 promoter activity, the 1584 bp full length (SEQ ID NO:2) and five 5′ unidirectional deletion fragments 1258 bp, 1002 bp, 690 bp, 448 bp, and 229 bp in length corresponding to SEQ ID NO:3, 4, 5, 6, and 7, respectively, were made by PCR amplification from the full length soybean PIP1 promoter contained in the original construct QC386. The same antisense primer QC386-A (SEQ ID NO:10) was used in the amplification by PCR of all the six PIP1 promoter fragments (SEQ ID NOs: 2, 3, 4, 5, 6, and 7) by pairing with different sense primers SEQ ID NOs:11, 12, 13, 14, 15, and 16, respectively. Each of the PCR amplified promoter DNA fragments was cloned into the GATEWAY® cloning ready TA cloning vector pCR8/GW/TOPO (Invitrogen) and clones with the correct orientation, relative to the GATEWAY® recombination sites attL1 and attL2, were selected by sequence confirmation. The promoter fragment in the right orientation was subsequently cloned into a GATEWAY® destination vector QC330 (SEQ ID NO:23) by GATEWAY® LR Clonase® reaction (Invitrogen) to place the promoter fragment in front of the reporter gene YFP. A 21 bp GATEWAY® recombination site attB2 (SEQ ID NO:47) was inserted between the promoter and the YFP reporter gene coding region as a result of the GATEWAY® cloning process. The maps of constructs QC386-2Y, 3Y, 4Y, 5Y, and 6Y containing the PIP1 promoter fragments SEQ ID NOs: 3, 4, 5, 6, and 7 are similar to QC386-1Y map and not shown.

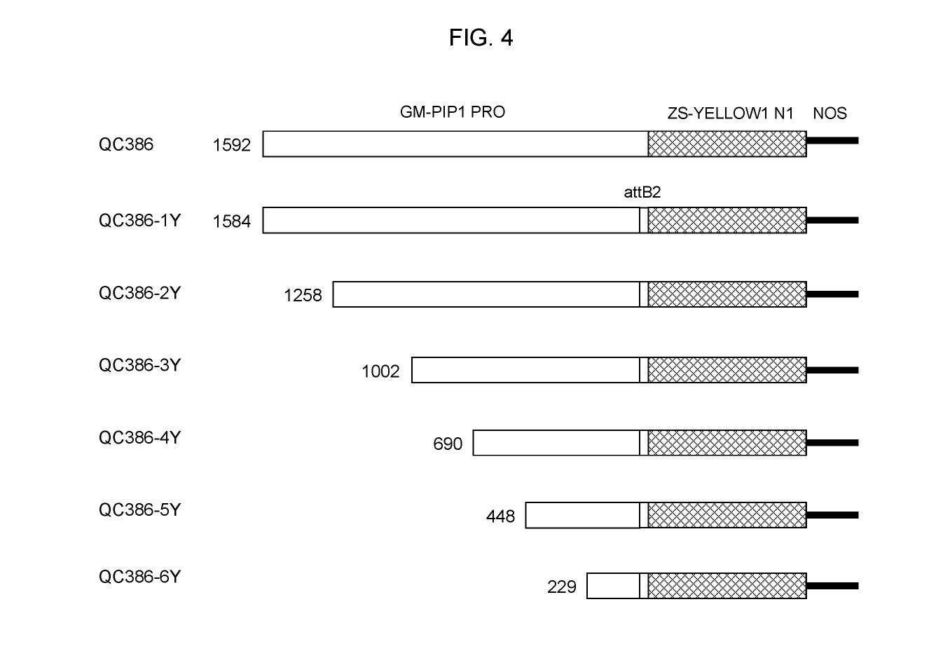

The PIP1:YFP promoter deletion constructs were delivered into germinating soybean cotyledons by gene gun bombardment for transient gene expression study. The full length PIP1 promoter in QC386 that does not have the attB2 site located between the promoter and the YFP gene was also included for transient expression analysis as a control. The seven PIP1 promoter fragments analyzed are schematically described in FIG. 4.

### Example 6

### Transient Expression Analysis of PIP1:YFP Constructs

The constructs containing the full length and truncated PIP1 promoter fragments (QC386, QC386-1Y, 2Y, 3Y, 4Y, 5Y, and 6Y) were tested by transiently expressing the ZS-YELLOW1 N1 (YFP) reporter gene in germinating soybean cotyledons. Soybean seeds were rinsed with 10% TWEEN® 20 in sterile water, surface sterilized with 70% ethanol for 2 minutes and then by 6% sodium hypochloride for 15 minutes. After rinsing the seeds were placed on wet filter paper in Petri dish to germinate for 4-6 days under light at 26° C. Green cotyledons were excised and placed inner side up on a 0.7% agar plate containing Murashige and Skoog media for particle gun bombardment. The DNA and gold particle mixtures were prepared similarly as described in EXAMPLE 4 except with more DNA (100 ng/μl). The bombardments were also carried out under similar parameters as described in EXAMPLE 4. YFP expression was checked under a Leica MZFLIII stereo microscope equipped with UV light source and appropriate light filters (Leica Microsystems Inc., Bannockburn, Ill.) and pictures were taken approximately 24 hours after bombardment with 8× magnification using a Leica DFC500 camera with settings as 0.60 gamma, 1.0×gain, 0.70 saturation, 61 color hue, 56 color saturation, and 0.51 second exposure.

The full length PIP1 promoter constructs QC386 and QC386-1Y had similar yellow fluorescence signals in transient expression assay by showing the small yellow dots in red background. Each dot represented a single cotyledon cell which appeared larger if the fluorescence signal was strong or smaller if the fluorescence signal was weak even under the same magnification. The signals are not as strong as the bright dots shown by the positive control construct pZSL90. QC386-1Y had fewer yellow dots probably due to the fluctuation of DNA actually delivered to the cotyledons in different bombardments since the attB2 site inserted between the PIP1 promoter and YFP gene did not seem to interfere with promoter activity and reporter gene expression for other deletion constructs. The deletion construct QC386-2Y showed strongest yellow fluorescence signals comparable to the positive control pZSL90. But more in depth study would be necessary to confirm if the deleted 326 bp 5′ end of the PIP1 promoter contained elements negatively affecting the promoter activity. Further deletions of the PIP1 promoter in QC386-3Y, 4Y, and 5Y resulted in further reductions of the promoter strength. The smallest deletion construct QC386-6Y also showed yellow dots, though smaller and very faint, suggesting that as short as 229 bp PIP1 promoter sequence upstream of the start codon ATG was long enough for the minimal expression of a reporter gene.

The data clearly indicates that all deletion constructs are functional as a constitutive promoter and as such SEQ ID NO: 2, 3, 5, 6, 7 are all functional fragments of SEQ ID NO:1.

### Example 7

### PIP1:YFP Expression in Stable Transgenic Soybean Plants

The stable expression of the fluorescent protein reporter gene ZS-YELLOW1 N1 (YFP) driven by the full length PIP1 promoter (SEQ ID NO:1, construct QC389) in transgenic soybean plants is described below.

YFP gene expression was tested at different stages of transgenic plant development for yellow fluorescence emission under a Leica MZFLIII stereo microscope equipped with appropriate fluorescent light filters. Yellow fluorescence was not detectable in globular and young heart stage somatic embryos during the suspension culture period of soybean transformation. YFP expression was first detected in differentiating somatic embryos placed on solid medium and then throughout later stages with strongest even expression in fully developed somatic embryos. The negative section of a positive embryo cluster emitted weak red color due to auto fluorescence from the chlorophyll contained in soybean green tissues including embryos. When transgenic plants regenerated, YFP expression was detected in most tissues tested, such as flower, leaf, stem, root, pod, and seed. Any green tissue such as leaf or stem negative for YFP expression would be red and any white tissue such as root and petal would be dull yellowish under the yellow fluorescent light filter.

A soybean flower consists of five sepals, five petals including one standard large upper petal, two large side petals, and two small lower petals called kneel to enclose ten stamens and one pistil. The pistil consists of a stigma, a style, and an ovary in which there are 2-4 ovules. A stamen consists of a filament, and an anther on its tip. The filaments of nine of the stamens are fused and elevated as a single structure with a posterior stamen remaining separate. Pollen grains reside inside anther chambers and are released during pollination the day before the fully opening of the flower. Fluorescence signals were detected in sepals and in sepals of both flower buds and open flowers and also in the stamens and pistil inside the flower. Fluorescence signals were detected in the inner lining of the pistil and also weakly in ovules.

Yellow fluorescence was detected weakly in both young trifoliate leaves of plantlet and in fully developed leaf of adult plant, in the cross and longitudinal sections of stem and moderately in root at TO plant stage. Fluorescence signals seemed to be primarily detected in the vascular bundles of stem and root.

Strong fluorescence signals were detected in developing seeds and also pods at all stages of the PIP1:YFP transgenic plants from young R3 pod of ˜5 mm long, to full R4 pod of ˜20 mm long, until elongated pods filled with R5, R6 seeds. Fluorescence signals were detected in both seed coat and embryos. The seed and pod development stages were defined according to descriptions in Fehr and Caviness, IWSRBC 80:1-12 (1977).

In conclusion, PIP1:YFP expression was detected moderately in most tissues throughout transgenic plant development indicating that the soybean PIP1 promoter is a moderate constitutive promoter.

What is claimed is:

1. A recombinant DNA construct comprising:
(a) a nucleotide sequence comprising the sequence set forth in SEQ ID NO:1, SEQ ID NO:2, SEQ ID NO:3, SEQ ID NO:4, SEQ ID NO:5, or SEQ ID NO:6, or SEQ ID NO: 49, or a functional fragment thereof; or,
(b) a full-length complement of (a); or,
(c) a nucleotide sequence comprising a sequence having at least 71% sequence identity, based on the Clustal V method of alignment with pairwise alignment default parameters (KTUPLE=2, GAP PENALTY=5, WINDOW=4 and DIAGONALS SAVED=4), when compared to the nucleotide sequence of (a);
wherein said nucleotide sequence is a promoter.

2. The recombinant DNA construct of claim 1, wherein the promoter is a constitutive promoter.

3. The recombinant DNA construct of claim 1, wherein said nucleotide sequence has at least 95% identity, based on the Clustal V method of alignment with pairwise alignment default parameters (KTUPLE=2, GAP PENALTY=5, WINDOW=4 and DIAGONALS SAVED=4), when compared to any one of the sequence set forth in SEQ ID NO:1, SEQ ID NO:2, SEQ ID NO:3, SEQ ID NO:4, SEQ ID NO:5, SEQ ID NO:6, or SEQ ID NO:49.

4. The recombinant DNA construct of claim 3, wherein said nucleotide sequence is SEQ ID NO: 49.

5. A vector comprising the recombinant DNA construct of claim 1.

6. A cell comprising the recombinant DNA construct of claim 1.

7. The cell of claim 6, wherein the cell is a plant cell.

8. A transgenic plant having stably incorporated into its genome the recombinant DNA construct of claim 1.

9. The transgenic plant of claim 8 wherein said plant is a dicot plant.

10. The transgenic plant of claim 8 wherein the plant is soybean.

11. A transgenic seed produced by the transgenic plant of claim 8.

12. The recombinant DNA construct according to claim 1, wherein the at least one heterologous nucleotide sequence codes for a gene selected from the group consisting of: a reporter gene, a selection marker, a disease resistance conferring gene, a herbicide resistance conferring gene, an insect resistance conferring gene; a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in cold resistance, a gene involved in heat resistance and a gene involved in salt resistance in plants.

13. The recombinant DNA construct according to claim 1, wherein the at least one heterologous nucleotide sequence encodes a protein selected from the group consisting of: a reporter protein, a selection marker, a protein conferring disease resistance, protein conferring herbicide resistance, protein conferring insect resistance; protein involved in carbohydrate metabolism, protein involved in fatty acid metabolism, protein involved in amino acid metabolism, protein involved in plant development, protein involved in plant growth regulation, protein involved in yield improvement, protein involved in drought resistance, protein involved in cold resistance, protein involved in heat resistance and protein involved in salt resistance in plants.

14. A method of expressing a coding sequence or a functional RNA in a plant comprising:
a) introducing the recombinant DNA construct of claim 1 into the plant, wherein the at least one heterologous nucleotide sequence comprises a coding sequence or a functional RNA;
b) growing the plant of step a); and
c) selecting a plant displaying expression of the coding sequence or the functional RNA of the recombinant DNA construct.

15. A method of transgenically altering a marketable plant trait, comprising:
a) introducing a recombinant DNA construct of claim 1 into the plant;
b) growing a fertile, mature plant resulting from step a); and
c) selecting a plant expressing the at least one heterologous nucleotide sequence in at least one plant tissue based on the altered marketable trait.

16. The method of claim 15 wherein the marketable trait is selected from the group consisting of: disease resistance, herbicide resistance, insect resistance carbohydrate metabolism, fatty acid metabolism, amino acid metabolism, plant development, plant growth regulation, yield improvement, drought resistance, cold resistance, heat resistance, and salt resistance.

17. A method for altering expression of at least one heterologous nucleic acid fragment in plant comprising:
(a) transforming a plant cell with the recombinant DNA construct of claim 1;
(b) growing fertile mature plants from transformed plant cell of step (a); and
(c) selecting plants containing the transformed plant cell wherein the expression of the heterologous nucleic acid fragment is increased or decreased.

18. The method of claim 17 wherein the plant is a soybean plant.

19. A method for expressing a yellow fluorescent protein ZS-YELLOW1 N1 in a host cell comprising:
(a) transforming a host cell with the recombinant DNA construct of claim 1; and,
(b) growing the transformed host cell under conditions that are suitable for expression of the recombinant DNA construct, wherein expression of the recombinant DNA construct results in production of increased levels of ZS-YELLOW1 N1 protein in the transformed host cell when compared to a corresponding non-transformed host cell.

20. A plant stably transformed with a recombinant DNA construct comprising a soybean constitutive promoter and a heterologous nucleic acid fragment operably linked to said constitutive promoter, wherein said constitutive promoter is a capable of controlling expression of said heterologous nucleic acid fragment in a plant cell, and further wherein said constitutive promoter comprises any of the sequences set forth in SEQ ID NO:1, SEQ ID NO:2, SEQ ID NO:3, SEQ ID NO:4, SEQ ID NO:5, SEQ ID NO:6, or SEQ ID NO:49.


SEQUENCE LISTING (50 sequences)

SEQ ID NO: 1 - DNA, 1592 bp, artificial sequence
  GM-PIP1 promoter 1592 bp, QC386
cccgggctaatcgagctggtactaaactaatgcatattaggtaatgcaaataaataataacgctcccaagaatattcaaatggtttcttttgctttttgcttaacgacttttgtatctctacgtattacttgagaaaaaaagctgctattattatccaactaaacaaatgaaagctacagttaaggacatggcctattaacaatattacgtagacttgatcattgtctcatccacgagatagaaacaaaatatataaaagggctcattatgcttatttagttcatcaagaagctaggaaaatgagtacgtagaatgaacatttaataatggacgtgagagaagttaatcgctgacagccatgtgccgaccatgttttttataaatgaaaagaaagaaatgttcgtatataataattaacggacacaagaaccttgttaataattatcattatctttttttttttgtttttattttccgaaaaacttgtttctccaatcattgatgtgtatttctattctctctccatttccaactcctgactgagaagtggatttcatatcaacattagcaattagtagaatactatcatctttcacgctacaaaacattggtactttggtaggtaaagatttgcaaacacgaatacgtaattaagaaaggttcatacacattcaatgattctggattcctaccttacgttatttgtttcgaaatacctagatgagagcatcttgttatttattactacatattaattttccctgtgtaccttgtcgtagtttaaatttattattttttcaatcataaataaatataagaaatatttttttcttaatataattttattttatatttaaaaataaatcataatttgaaagagctacaaatttataccacatgtgggaagtattgttggtttctccaaccatacttattgagaataacttgaatttatattcaacgtattaattgcttcacctttaacgtgccaaaataataataataaaaaacttaaaactactgtattaatcgcgtgtggttgaatggaggcaaattctattctaaaaaagaaaaagcattaacaaaaggagaaaagaaaaactgttgacacctgacagcagtaacagggaactgggaagtagcagtaggagtatttgcgtgttggtttccaactctggaatccaccgtgccaaactgcgaatgcaggagaaatcgacacgtgtccatttgcaggcgcgagttgaacgtgacaatgcaccaccgcccagcatcgaacgcagccaaggaccacgtcgaaaccacagtaatccacgttccagtgctgcgcggaacatggtcggtctttctaggagtggttggaatcacgccagctaggacaaaccccatcaatcattggtcattatcaaacaaaacatttcaaaaattcaacatattacgcctcgggacccacctcccactacacctcaccctcacttctattaactcgaacacattcgggttataaatccgcaaccctccttctcactcactcactcactcactcactcactcgcaagcaaaaagaaagaatcccaggcgaggagaaccatgg

SEQ ID NO: 2 - DNA, 1584 bp, artificial sequence
  GM-PIP1 promoter 1584 bp, QC386-1
gggctaatcgagctggtactaaactaatgcatattaggtaatgcaaataaataataacgctcccaagaatattcaaatggtttcttttgctttttgcttaacgacttttgtatctctacgtattacttgagaaaaaaagctgctattattatccaactaaacaaatgaaagctacagttaaggacatggcctattaacaatattacgtagacttgatcattgtctcatccacgagatagaaacaaaatatataaaagggctcattatgcttatttagttcatcaagaagctaggaaaatgagtacgtagaatgaacatttaataatggacgtgagagaagttaatcgctgacagccatgtgccgaccatgttttttataaatgaaaagaaagaaatgttcgtatataataattaacggacacaagaaccttgttaataattatcattatctttttttttttgtttttattttccgaaaaacttgtttctccaatcattgatgtgtatttctattctctctccatttccaactcctgactgagaagtggatttcatatcaacattagcaattagtagaatactatcatctttcacgctacaaaacattggtactttggtaggtaaagatttgcaaacacgaatacgtaattaagaaaggttcatacacattcaatgattctggattcctaccttacgttatttgtttcgaaatacctagatgagagcatcttgttatttattactacatattaattttccctgtgtaccttgtcgtagtttaaatttattattttttcaatcataaataaatataagaaatatttttttcttaatataattttattttatatttaaaaataaatcataatttgaaagagctacaaatttataccacatgtgggaagtattgttggtttctccaaccatacttattgagaataacttgaatttatattcaacgtattaattgcttcacctttaacgtgccaaaataataataataaaaaacttaaaactactgtattaatcgcgtgtggttgaatggaggcaaattctattctaaaaaagaaaaagcattaacaaaaggagaaaagaaaaactgttgacacctgacagcagtaacagggaactgggaagtagcagtaggagtatttgcgtgttggtttccaactctggaatccaccgtgccaaactgcgaatgcaggagaaatcgacacgtgtccatttgcaggcgcgagttgaacgtgacaatgcaccaccgcccagcatcgaacgcagccaaggaccacgtcgaaaccacagtaatccacgttccagtgctgcgcggaacatggtcggtctttctaggagtggttggaatcacgccagctaggacaaaccccatcaatcattggtcattatcaaacaaaacatttcaaaaattcaacatattacgcctcgggacccacctcccactacacctcaccctcacttctattaactcgaacacattcgggttataaatccgcaaccctccttctcactcactcactcactcactcactcactcgcaagcaaaaagaaagaatcccaggcgaggagaac

SEQ ID NO: 3 - DNA, 1258 bp, artificial sequence
  GM-PIP1 promoter 1258 bp, QC386-2
ggacgtgagagaagttaatcgctgacagccatgtgccgaccatgttttttataaatgaaaagaaagaaatgttcgtatataataattaacggacacaagaaccttgttaataattatcattatctttttttttttgtttttattttccgaaaaacttgtttctccaatcattgatgtgtatttctattctctctccatttccaactcctgactgagaagtggatttcatatcaacattagcaattagtagaatactatcatctttcacgctacaaaacattggtactttggtaggtaaagatttgcaaacacgaatacgtaattaagaaaggttcatacacattcaatgattctggattcctaccttacgttatttgtttcgaaatacctagatgagagcatcttgttatttattactacatattaattttccctgtgtaccttgtcgtagtttaaatttattattttttcaatcataaataaatataagaaatatttttttcttaatataattttattttatatttaaaaataaatcataatttgaaagagctacaaatttataccacatgtgggaagtattgttggtttctccaaccatacttattgagaataacttgaatttatattcaacgtattaattgcttcacctttaacgtgccaaaataataataataaaaaacttaaaactactgtattaatcgcgtgtggttgaatggaggcaaattctattctaaaaaagaaaaagcattaacaaaaggagaaaagaaaaactgttgacacctgacagcagtaacagggaactgggaagtagcagtaggagtatttgcgtgttggtttccaactctggaatccaccgtgccaaactgcgaatgcaggagaaatcgacacgtgtccatttgcaggcgcgagttgaacgtgacaatgcaccaccgcccagcatcgaacgcagccaaggaccacgtcgaaaccacagtaatccacgttccagtgctgcgcggaacatggtcggtctttctaggagtggttggaatcacgccagctaggacaaaccccatcaatcattggtcattatcaaacaaaacatttcaaaaattcaacatattacgcctcgggacccacctcccactacacctcaccctcacttctattaactcgaacacattcgggttataaatccgcaaccctccttctcactcactcactcactcactcactcactcgcaagcaaaaagaaagaatcccaggcgaggagaac

SEQ ID NO: 4 - DNA, 1002 bp, artificial sequence
  GM-PIP1 promoter 1002 bp, QC386-3
atcatctttcacgctacaaaacattggtactttggtaggtaaagatttgcaaacacgaatacgtaattaagaaaggttcatacacattcaatgattctggattcctaccttacgttatttgtttcgaaatacctagatgagagcatcttgttatttattactacatattaattttccctgtgtaccttgtcgtagtttaaatttattattttttcaatcataaataaatataagaaatatttttttcttaatataattttattttatatttaaaaataaatcataatttgaaagagctacaaatttataccacatgtgggaagtattgttggtttctccaaccatacttattgagaataacttgaatttatattcaacgtattaattgcttcacctttaacgtgccaaaataataataataaaaaacttaaaactactgtattaatcgcgtgtggttgaatggaggcaaattctattctaaaaaagaaaaagcattaacaaaaggagaaaagaaaaactgttgacacctgacagcagtaacagggaactgggaagtagcagtaggagtatttgcgtgttggtttccaactctggaatccaccgtgccaaactgcgaatgcaggagaaatcgacacgtgtccatttgcaggcgcgagttgaacgtgacaatgcaccaccgcccagcatcgaacgcagccaaggaccacgtcgaaaccacagtaatccacgttccagtgctgcgcggaacatggtcggtctttctaggagtggttggaatcacgccagctaggacaaaccccatcaatcattggtcattatcaaacaaaacatttcaaaaattcaacatattacgcctcgggacccacctcccactacacctcaccctcacttctattaactcgaacacattcgggttataaatccgcaaccctccttctcactcactcactcactcactcactcactcgcaagcaaaaagaaagaatcccaggcgaggagaac

SEQ ID NO: 5 - DNA, 690 bp, artificial sequence
  GM-PIP1 promoter 690 bp, QC386-4
catgtgggaagtattgttggtttctccaaccatacttattgagaataacttgaatttatattcaacgtattaattgcttcacctttaacgtgccaaaataataataataaaaaacttaaaactactgtattaatcgcgtgtggttgaatggaggcaaattctattctaaaaaagaaaaagcattaacaaaaggagaaaagaaaaactgttgacacctgacagcagtaacagggaactgggaagtagcagtaggagtatttgcgtgttggtttccaactctggaatccaccgtgccaaactgcgaatgcaggagaaatcgacacgtgtccatttgcaggcgcgagttgaacgtgacaatgcaccaccgcccagcatcgaacgcagccaaggaccacgtcgaaaccacagtaatccacgttccagtgctgcgcggaacatggtcggtctttctaggagtggttggaatcacgccagctaggacaaaccccatcaatcattggtcattatcaaacaaaacatttcaaaaattcaacatattacgcctcgggacccacctcccactacacctcaccctcacttctattaactcgaacacattcgggttataaatccgcaaccctccttctcactcactcactcactcactcactcactcgcaagcaaaaagaaagaatcccaggcgaggagaac

SEQ ID NO: 6 - DNA, 448 bp, artificial sequence
  GM-PIP1 promoter 448 bp, QC386-5
gtagcagtaggagtatttgcgtgttggtttccaactctggaatccaccgtgccaaactgcgaatgcaggagaaatcgacacgtgtccatttgcaggcgcgagttgaacgtgacaatgcaccaccgcccagcatcgaacgcagccaaggaccacgtcgaaaccacagtaatccacgttccagtgctgcgcggaacatggtcggtctttctaggagtggttggaatcacgccagctaggacaaaccccatcaatcattggtcattatcaaacaaaacatttcaaaaattcaacatattacgcctcgggacccacctcccactacacctcaccctcacttctattaactcgaacacattcgggttataaatccgcaaccctccttctcactcactcactcactcactcactcactcgcaagcaaaaagaaagaatcccaggcgaggagaac

SEQ ID NO: 7 - DNA, 229 bp, artificial sequence
  GM-PIP1 promoter 229 bp, QC386-6
ggaatcacgccagctaggacaaaccccatcaatcattggtcattatcaaacaaaacatttcaaaaattcaacatattacgcctcgggacccacctcccactacacctcaccctcacttctattaactcgaacacattcgggttataaatccgcaaccctccttctcactcactcactcactcactcactcactcgcaagcaaaaagaaagaatcccaggcgaggagaac

SEQ ID NO: 8 - DNA, 38 bp, artificial sequence
  primer, PSO332986Xma
ataatcccgggctaatcgagctggtactaaactaatgc

SEQ ID NO: 9 - DNA, 34 bp, artificial sequence
  primer, PSO332986Nco
tgattccatggttctcctcgcctgggattctttc

SEQ ID NO: 10 - DNA, 24 bp, artificial sequence
  primer, QC386-A
gttctcctcgcctgggattctttc

SEQ ID NO: 11 - DNA, 29 bp, artificial sequence
  primer, QC386-S1
gggctaatcgagctggtactaaactaatg

SEQ ID NO: 12 - DNA, 26 bp, artificial sequence
  primer, QC386-S2
ggacgtgagagaagttaatcgctgac

SEQ ID NO: 13 - DNA, 27 bp, artificial sequence
  primer, QC386-S3
atcatctttcacgctacaaaacattgg

SEQ ID NO: 14 - DNA, 27 bp, artificial sequence
  primer, PSO332986S2
catgtgggaagtattgttggtttctcc

SEQ ID NO: 15 - DNA, 27 bp, artificial sequence
  primer, QC386-S5
gtagcagtaggagtatttgcgtgttgg

SEQ ID NO: 16 - DNA, 24 bp, artificial sequence
  primer, QC386-S6
ggaatcacgccagctaggacaaac

SEQ ID NO: 17 - DNA, 1247 bp, Glycine max
ctcactcactcactcactcactcactcactcgcaagcaaaaagaaagaatcccaggcgaggagaaagatggaggggaaggagcaggatgtgtcgttgggagcgaacaagttccccgagagacagccaattgggacggcggcgcagagccaagacgacggcaaggactaccaggagccggcgccggcgccgctggttgacccgacggagtttacgtcatggtcgttttacagagcagggatagcagagtttgtggccacttttctgtttctctacatcactgtcttaaccgttatgggagtcgccggggctaagtctaagtgtagtaccgttgggattcaaggaatcgcttgggccttcggtggcatgatcttcgccctcgtttactgcaccgctggcatctcagggggacacataaacccggcggtgacatttgggctgtttttggcgaggaagttgtcgttgcccagggcgattttctacatcgtgatgcaatgcttgggtgctatttgtggcgctggcgtggtgaagggtttcgaggggaaaacaaaatacggtgcgttgaatggtggtgccaactttgttgcccctggttacaccaagggtgatggtcttggtgctgagattgttggcactttcatccttgtttacaccgttttctccgccaccgatgccaaacgtagcgccagagactcccacgtccccattttggcacccttgccaattgggttcgctgtgttcttggttcacttggcaaccatccccatcaccggaactggtatcaaccctgctcgtagtcttggtgctgctatcatcttcaacaaggaccttggttgggatgaacactggatcttctgggtgggaccattcatcggtgcagctcttgcagcactctaccaccaggtcgtaatcagggccattcccttcaagtccaagtgattcaatcaaacggttcatgcttaatcaagttgggaacaacaacaacaacaaaaatcaagccaatgtttgtgggttttggtttcatttcattaagatgatctgtttatctcttttcttctttttaaaatttaaagtctttgtattttgtatgtaaagatgtaaaattatgattattaggtggtgcatgtgtcgcgtcatgggccaatgttatcctctgcttttaagttggaagaggcccaactcatgtgtgatgtacggctgtgattgtgtaatttaatttgcaaaatcaaaaataacaccagagtcatat

SEQ ID NO: 18 - protein, 289 aa, Glycine max
MEGKEQDVSLGANKFPERQPIGTAAQSQDDGKDYQEPAPAPLVDPTEFTSWSFYRAGIAEFVATFLFLYITVLTVMGVAGAKSKCSTVGIQGIAWAFGGMIFALVYCTAGISGGHINPAVTFGLFLARKLSLPRAIFYIVMQCLGAICGAGVVKGFEGKTKYGALNGGANFVAPGYTKGDGLGAEIVGTFILVYTVFSATDAKRSARDSHVPILAPLPIGFAVFLVHLATIPITGTGINPARSLGAAIIFNKDLGWDEHWIFWVGPFIGAALAALYHQVVIRAIPFKSK

SEQ ID NO: 19 - DNA, 4869 bp, artificial sequence
  plasmid QC386, 4869 bp
gggctaatcgagctggtactaaactaatgcatattaggtaatgcaaataaataataacgctcccaagaatattcaaatggtttcttttgctttttgcttaacgacttttgtatctctacgtattacttgagaaaaaaagctgctattattatccaactaaacaaatgaaagctacagttaaggacatggcctattaacaatattacgtagacttgatcattgtctcatccacgagatagaaacaaaatatataaaagggctcattatgcttatttagttcatcaagaagctaggaaaatgagtacgtagaatgaacatttaataatggacgtgagagaagttaatcgctgacagccatgtgccgaccatgttttttataaatgaaaagaaagaaatgttcgtatataataattaacggacacaagaaccttgttaataattatcattatctttttttttttgtttttattttccgaaaaacttgtttctccaatcattgatgtgtatttctattctctctccatttccaactcctgactgagaagtggatttcatatcaacattagcaattagtagaatactatcatctttcacgctacaaaacattggtactttggtaggtaaagatttgcaaacacgaatacgtaattaagaaaggttcatacacattcaatgattctggattcctaccttacgttatttgtttcgaaatacctagatgagagcatcttgttatttattactacatattaattttccctgtgtaccttgtcgtagtttaaatttattattttttcaatcataaataaatataagaaatatttttttcttaatataattttattttatatttaaaaataaatcataatttgaaagagctacaaatttataccacatgtgggaagtattgttggtttctccaaccatacttattgagaataacttgaatttatattcaacgtattaattgcttcacctttaacgtgccaaaataataataataaaaaacttaaaactactgtattaatcgcgtgtggttgaatggaggcaaattctattctaaaaaagaaaaagcattaacaaaaggagaaaagaaaaactgttgacacctgacagcagtaacagggaactgggaagtagcagtaggagtatttgcgtgttggtttccaactctggaatccaccgtgccaaactgcgaatgcaggagaaatcgacacgtgtccatttgcaggcgcgagttgaacgtgacaatgcaccaccgcccagcatcgaacgcagccaaggaccacgtcgaaaccacagtaatccacgttccagtgctgcgcggaacatggtcggtctttctaggagtggttggaatcacgccagctaggacaaaccccatcaatcattggtcattatcaaacaaaacatttcaaaaattcaacatattacgcctcgggacccacctcccactacacctcaccctcacttctattaactcgaacacattcgggttataaatccgcaaccctccttctcactcactcactcactcactcactcactcgcaagcaaaaagaaagaatcccaggcgaggagaaccatggcccacagcaagcacggcctgaaggaggagatgaccatgaagtaccacatggagggctgcgtgaacggccacaagttcgtgatcaccggcgagggcatcggctaccccttcaagggcaagcagaccatcaacctgtgcgtgatcgagggcggccccctgcccttcagcgaggacatcctgagcgccggcttcaagtacggcgaccggatcttcaccgagtacccccaggacatcgtggactacttcaagaacagctgccccgccggctacacctggggccggagcttcctgttcgaggacggcgccgtgtgcatctgtaacgtggacatcaccgtgagcgtgaaggagaactgcatctaccacaagagcatcttcaacggcgtgaacttccccgccgacggccccgtgatgaagaagatgaccaccaactgggaggccagctgcgagaagatcatgcccgtgcctaagcagggcatcctgaagggcgacgtgagcatgtacctgctgctgaaggacggcggccggtaccggtgccagttcgacaccgtgtacaaggccaagagcgtgcccagcaagatgcccgagtggcacttcatccagcacaagctgctgcgggaggaccggagcgacgccaagaaccagaagtggcagctgaccgagcacgccatcgccttccccagcgccctggcctgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattctagtggccggcccagctgatatccatcacactggcggccgcactcgactgaattggttccggcgccagcctgcttttttgtacaaagttggcattataaaaaagcattgcttatcaatttgttgcaacgaacaggtcactatcagtcaaaataaaatcattatttggggcccgagcttaagtaactaactaacaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcttagtagttagacgtccccgagatccatgctagcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacggggcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcagctggatggcaaataatgattttattttgactgatagtgacctgttcgttgcaacaaattgataagcaatgctttcttataatgccaactttgtacaagaaagctgggtctagatatctcgaccc

SEQ ID NO: 20 - DNA, 8409 bp, artificial sequence
  plasmid QC324i
atcaaccactttgtacaagaaagctgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccagatctttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacggatcctaactcaaaatccacacattatacgagccggaagcataaagtgtaaagcctggggtgcctaatgcggccgccaatatgactggatatgttgtgttttacagtattatgtagtctgttttttatgcaaaatctaatttaatatattgatatttatatcattttacgtttctcgttcagcttttttgtacaaacttgttgatggggttaacatatcataacttcgtataatgtatgctatacgaagttataggcctggatcttcgaggtcgagcggccgcagatttaggtgacactatagaatatgcatcactagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgtttgactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatattcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccatcagatcgaattaacatatcataacttcgtataatgtatgctatacgaagttataggcctggatccactagttctagagcggccgctcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccggtaccc

SEQ ID NO: 21 - DNA, 9394 bp, artificial sequence
  plasmid QC389
gggctaatcgagctggtactaaactaatgcatattaggtaatgcaaataaataataacgctcccaagaatattcaaatggtttcttttgctttttgcttaacgacttttgtatctctacgtattacttgagaaaaaaagctgctattattatccaactaaacaaatgaaagctacagttaaggacatggcctattaacaatattacgtagacttgatcattgtctcatccacgagatagaaacaaaatatataaaagggctcattatgcttatttagttcatcaagaagctaggaaaatgagtacgtagaatgaacatttaataatggacgtgagagaagttaatcgctgacagccatgtgccgaccatgttttttataaatgaaaagaaagaaatgttcgtatataataattaacggacacaagaaccttgttaataattatcattatctttttttttttgtttttattttccgaaaaacttgtttctccaatcattgatgtgtatttctattctctctccatttccaactcctgactgagaagtggatttcatatcaacattagcaattagtagaatactatcatctttcacgctacaaaacattggtactttggtaggtaaagatttgcaaacacgaatacgtaattaagaaaggttcatacacattcaatgattctggattcctaccttacgttatttgtttcgaaatacctagatgagagcatcttgttatttattactacatattaattttccctgtgtaccttgtcgtagtttaaatttattattttttcaatcataaataaatataagaaatatttttttcttaatataattttattttatatttaaaaataaatcataatttgaaagagctacaaatttataccacatgtgggaagtattgttggtttctccaaccatacttattgagaataacttgaatttatattcaacgtattaattgcttcacctttaacgtgccaaaataataataataaaaaacttaaaactactgtattaatcgcgtgtggttgaatggaggcaaattctattctaaaaaagaaaaagcattaacaaaaggagaaaagaaaaactgttgacacctgacagcagtaacagggaactgggaagtagcagtaggagtatttgcgtgttggtttccaactctggaatccaccgtgccaaactgcgaatgcaggagaaatcgacacgtgtccatttgcaggcgcgagttgaacgtgacaatgcaccaccgcccagcatcgaacgcagccaaggaccacgtcgaaaccacagtaatccacgttccagtgctgcgcggaacatggtcggtctttctaggagtggttggaatcacgccagctaggacaaaccccatcaatcattggtcattatcaaacaaaacatttcaaaaattcaacatattacgcctcgggacccacctcccactacacctcaccctcacttctattaactcgaacacattcgggttataaatccgcaaccctccttctcactcactcactcactcactcactcactcgcaagcaaaaagaaagaatcccaggcgaggagaaccatggcccacagcaagcacggcctgaaggaggagatgaccatgaagtaccacatggagggctgcgtgaacggccacaagttcgtgatcaccggcgagggcatcggctaccccttcaagggcaagcagaccatcaacctgtgcgtgatcgagggcggccccctgcccttcagcgaggacatcctgagcgccggcttcaagtacggcgaccggatcttcaccgagtacccccaggacatcgtggactacttcaagaacagctgccccgccggctacacctggggccggagcttcctgttcgaggacggcgccgtgtgcatctgtaacgtggacatcaccgtgagcgtgaaggagaactgcatctaccacaagagcatcttcaacggcgtgaacttccccgccgacggccccgtgatgaagaagatgaccaccaactgggaggccagctgcgagaagatcatgcccgtgcctaagcagggcatcctgaagggcgacgtgagcatgtacctgctgctgaaggacggcggccggtaccggtgccagttcgacaccgtgtacaaggccaagagcgtgcccagcaagatgcccgagtggcacttcatccagcacaagctgctgcgggaggaccggagcgacgccaagaaccagaagtggcagctgaccgagcacgccatcgccttccccagcgccctggcctgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattctagtggccggcccagctgatatccatcacactggcggccgcactcgactgaattggttccggcgccagcctgcttttttgtacaaacttgttgatggggttaacatatcataacttcgtataatgtatgctatacgaagttataggcctggatcttcgaggtcgagcggccgcagatttaggtgacactatagaatatgcatcactagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgtttgactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatattcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccatcagatcgaattaacatatcataacttcgtataatgtatgctatacgaagttataggcctggatccactagttctagagcggccgctcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccggtacccatcaaccactttgtacaagaaagctgggtctagatatctcgaccc

SEQ ID NO: 22 - DNA, 4401 bp, artificial sequence
  plasmid QC386-1
gggctaatcgagctggtactaaactaatgcatattaggtaatgcaaataaataataacgctcccaagaatattcaaatggtttcttttgctttttgcttaacgacttttgtatctctacgtattacttgagaaaaaaagctgctattattatccaactaaacaaatgaaagctacagttaaggacatggcctattaacaatattacgtagacttgatcattgtctcatccacgagatagaaacaaaatatataaaagggctcattatgcttatttagttcatcaagaagctaggaaaatgagtacgtagaatgaacatttaataatggacgtgagagaagttaatcgctgacagccatgtgccgaccatgttttttataaatgaaaagaaagaaatgttcgtatataataattaacggacacaagaaccttgttaataattatcattatctttttttttttgtttttattttccgaaaaacttgtttctccaatcattgatgtgtatttctattctctctccatttccaactcctgactgagaagtggatttcatatcaacattagcaattagtagaatactatcatctttcacgctacaaaacattggtactttggtaggtaaagatttgcaaacacgaatacgtaattaagaaaggttcatacacattcaatgattctggattcctaccttacgttatttgtttcgaaatacctagatgagagcatcttgttatttattactacatattaattttccctgtgtaccttgtcgtagtttaaatttattattttttcaatcataaataaatataagaaatatttttttcttaatataattttattttatatttaaaaataaatcataatttgaaagagctacaaatttataccacatgtgggaagtattgttggtttctccaaccatacttattgagaataacttgaatttatattcaacgtattaattgcttcacctttaacgtgccaaaataataataataaaaaacttaaaactactgtattaatcgcgtgtggttgaatggaggcaaattctattctaaaaaagaaaaagcattaacaaaaggagaaaagaaaaactgttgacacctgacagcagtaacagggaactgggaagtagcagtaggagtatttgcgtgttggtttccaactctggaatccaccgtgccaaactgcgaatgcaggagaaatcgacacgtgtccatttgcaggcgcgagttgaacgtgacaatgcaccaccgcccagcatcgaacgcagccaaggaccacgtcgaaaccacagtaatccacgttccagtgctgcgcggaacatggtcggtctttctaggagtggttggaatcacgccagctaggacaaaccccatcaatcattggtcattatcaaacaaaacatttcaaaaattcaacatattacgcctcgggacccacctcccactacacctcaccctcacttctattaactcgaacacattcgggttataaatccgcaaccctccttctcactcactcactcactcactcactcactcgcaagcaaaaagaaagaatcccaggcgaggagaacaagggcgaattcgacccagctttcttgtacaaagttggcattataaaaaataattgctcatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgcctcctctagaccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgaattcgccctt

SEQ ID NO: 23 - DNA, 5286 bp, artificial sequence
  plasmid QC330
atcaacaagtttgtacaaaaaagctgaacgagaaacgtaaaatgatataaatatcaatatattaaattagattttgcataaaaaacagactacataatactgtaaaacacaacatatccagtcatattggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatgggatccatggcccacagcaagcacggcctgaaggaggagatgaccatgaagtaccacatggagggctgcgtgaacggccacaagttcgtgatcaccggcgagggcatcggctaccccttcaagggcaagcagaccatcaacctgtgcgtgatcgagggcggccccctgcccttcagcgaggacatcctgagcgccggcttcaagtacggcgaccggatcttcaccgagtacccccaggacatcgtggactacttcaagaacagctgccccgccggctacacctggggccggagcttcctgttcgaggacggcgccgtgtgcatctgtaacgtggacatcaccgtgagcgtgaaggagaactgcatctaccacaagagcatcttcaacggcgtgaacttccccgccgacggccccgtgatgaagaagatgaccaccaactgggaggccagctgcgagaagatcatgcccgtgcctaagcagggcatcctgaagggcgacgtgagcatgtacctgctgctgaaggacggcggccggtaccggtgccagttcgacaccgtgtacaaggccaagagcgtgcccagcaagatgcccgagtggcacttcatccagcacaagctgctgcgggaggaccggagcgacgccaagaaccagaagtggcagctgaccgagcacgccatcgccttccccagcgccctggcctgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattctagtggccggcccagctgatatccatcacactggcggccgctcgagttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgtcgaggcctcacgtgttaacaagcttgcatgcctgcaggttt

SEQ ID NO: 24 - DNA, 5242 bp, artificial sequence
  plasmid QC386-1Y
gggctaatcgagctggtactaaactaatgcatattaggtaatgcaaataaataataacgctcccaagaatattcaaatggtttcttttgctttttgcttaacgacttttgtatctctacgtattacttgagaaaaaaagctgctattattatccaactaaacaaatgaaagctacagttaaggacatggcctattaacaatattacgtagacttgatcattgtctcatccacgagatagaaacaaaatatataaaagggctcattatgcttatttagttcatcaagaagctaggaaaatgagtacgtagaatgaacatttaataatggacgtgagagaagttaatcgctgacagccatgtgccgaccatgttttttataaatgaaaagaaagaaatgttcgtatataataattaacggacacaagaaccttgttaataattatcattatctttttttttttgtttttattttccgaaaaacttgtttctccaatcattgatgtgtatttctattctctctccatttccaactcctgactgagaagtggatttcatatcaacattagcaattagtagaatactatcatctttcacgctacaaaacattggtactttggtaggtaaagatttgcaaacacgaatacgtaattaagaaaggttcatacacattcaatgattctggattcctaccttacgttatttgtttcgaaatacctagatgagagcatcttgttatttattactacatattaattttccctgtgtaccttgtcgtagtttaaatttattattttttcaatcataaataaatataagaaatatttttttcttaatataattttattttatatttaaaaataaatcataatttgaaagagctacaaatttataccacatgtgggaagtattgttggtttctccaaccatacttattgagaataacttgaatttatattcaacgtattaattgcttcacctttaacgtgccaaaataataataataaaaaacttaaaactactgtattaatcgcgtgtggttgaatggaggcaaattctattctaaaaaagaaaaagcattaacaaaaggagaaaagaaaaactgttgacacctgacagcagtaacagggaactgggaagtagcagtaggagtatttgcgtgttggtttccaactctggaatccaccgtgccaaactgcgaatgcaggagaaatcgacacgtgtccatttgcaggcgcgagttgaacgtgacaatgcaccaccgcccagcatcgaacgcagccaaggaccacgtcgaaaccacagtaatccacgttccagtgctgcgcggaacatggtcggtctttctaggagtggttggaatcacgccagctaggacaaaccccatcaatcattggtcattatcaaacaaaacatttcaaaaattcaacatattacgcctcgggacccacctcccactacacctcaccctcacttctattaactcgaacacattcgggttataaatccgcaaccctccttctcactcactcactcactcactcactcactcgcaagcaaaaagaaagaatcccaggcgaggagaacaagggcgaattcgacccagctttcttgtacaaagtggttgatgggatccatggcccacagcaagcacggcctgaaggaggagatgaccatgaagtaccacatggagggctgcgtgaacggccacaagttcgtgatcaccggcgagggcatcggctaccccttcaagggcaagcagaccatcaacctgtgcgtgatcgagggcggccccctgcccttcagcgaggacatcctgagcgccggcttcaagtacggcgaccggatcttcaccgagtacccccaggacatcgtggactacttcaagaacagctgccccgccggctacacctggggccggagcttcctgttcgaggacggcgccgtgtgcatctgtaacgtggacatcaccgtgagcgtgaaggagaactgcatctaccacaagagcatcttcaacggcgtgaacttccccgccgacggccccgtgatgaagaagatgaccaccaactgggaggccagctgcgagaagatcatgcccgtgcctaagcagggcatcctgaagggcgacgtgagcatgtacctgctgctgaaggacggcggccggtaccggtgccagttcgacaccgtgtacaaggccaagagcgtgcccagcaagatgcccgagtggcacttcatccagcacaagctgctgcgggaggaccggagcgacgccaagaaccagaagtggcagctgaccgagcacgccatcgccttccccagcgccctggcctgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattctagtggccggcccagctgatatccatcacactggcggccgctcgagttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgtcgaggcctcacgtgttaacaagcttgcatgcctgcaggtttatcaacaagtttgtacaaaaaagcaggctccgaattcgccctt

SEQ ID NO: 25 - DNA, 26 bp, artificial sequence
  Sams-L primer
gaccaagacacactcgttcatatatc

SEQ ID NO: 26 - DNA, 25 bp, artificial sequence
  Sams-L2 primer
tctgctgctcaatgtttacaaggac

SEQ ID NO: 27 - DNA, 18 bp, artificial sequence
  primer, PSO332986F
tggtgcatgtgtcgcgtc

SEQ ID NO: 28 - DNA, 20 bp, artificial sequence
  primer, PSO332986R
catcacacatgagttgggcc

SEQ ID NO: 29 - DNA, 24 bp, artificial sequence
  ATPS sense primer
catgattgggagaaaccttaagct

SEQ ID NO: 30 - DNA, 20 bp, artificial sequence
  ATPS antisense primer
agattgggccagaggatcct

SEQ ID NO: 31 - DNA, 19 bp, artificial sequence
  primer, PSO332986JK-S
gccagagactcccacgtcc

SEQ ID NO: 32 - DNA, 24 bp, artificial sequence
  primer, PSO332986JK-A
ttacacaatcacagccgtacatca

SEQ ID NO: 33 - DNA, 22 bp, artificial sequence
  SAMS forward primer(SAMS-76F)
aggcttgttgtgcagtttttga

SEQ ID NO: 34 - DNA, 22 bp, artificial sequence
  FAM labeled ALS probe(ALS-100T)
ccacacaacacaatggcggcca

SEQ ID NO: 35 - DNA, 22 bp, artificial sequence
  ALS reverse primer(ALS-163R
ggaagaagagaatcgggtggtt

SEQ ID NO: 36 - DNA, 20 bp, artificial sequence
  YFP forward primer(YFP-67F)
aacggccacaagttcgtgat

SEQ ID NO: 37 - DNA, 20 bp, artificial sequence
  FAM labeled YFP probe(YFP-88T)
accggcgagggcatcggcta

SEQ ID NO: 38 - DNA, 20 bp, artificial sequence
  YFP reverse primer (YFP-130R)
cttcaagggcaagcagacca

SEQ ID NO: 39 - DNA, 24 bp, artificial sequence
  HSP forward primer (HSP-F1)
caaacttgacaaagccacaactct

SEQ ID NO: 40 - DNA, 20 bp, artificial sequence
  VIC labeled HSP probe (HSP probe)
ctctcatctcatataaatac

SEQ ID NO: 41 - DNA, 21 bp, artificial sequence
  HSP reverse primer (HSP-R1)
ggagaaattggtgtcgtggaa

SEQ ID NO: 42 - DNA, 100 bp, artificial sequence
  attL1
caaataatgattttattttgactgatagtgacctgttcgttgcaacaaattgataagcaatgcttttttataatgccaactttgtacaaaaaagcaggct

SEQ ID NO: 43 - DNA, 100 bp, artificial sequence
  attL2
caaataatgattttattttgactgatagtgacctgttcgttgcaacaaattgataagcaatgctttcttataatgccaactttgtacaagaaagctgggt

SEQ ID NO: 44 - DNA, 125 bp, artificial sequence
  attR1
acaagtttgtacaaaaaagctgaacgagaaacgtaaaatgatataaatatcaatatattaaattagattttgcataaaaaacagactacataatactgtaaaacacaacatatccagtcatattg

SEQ ID NO: 45 - DNA, 125 bp, artificial sequence
  attR2
accactttgtacaagaaagctgaacgagaaacgtaaaatgatataaatatcaatatattaaattagattttgcataaaaaacagactacataatactgtaaaacacaacatatccagtcactatg

SEQ ID NO: 46 - DNA, 21 bp, artificial sequence
  attB1
caagtttgtacaaaaaagcag

SEQ ID NO: 47 - DNA, 21 bp, artificial sequence
  attB2
cagctttcttgtacaaagtgg

SEQ ID NO: 48 - DNA, 1378 bp, artificial sequence
  NCBI accession AK246127.1, 1378 bp
ggcaaccctccttctcactcactcactcactcactcactcactcgcaagcaaaaagaaagaatcccaggcgaggagaaagatggaggggaaggagcaggatgtgtcgttgggagcgaacaagttccccgagagacagccaattgggacggcggcgcagagccaagacgacggcaaggactaccaggagccggcgccggcgccgctggttgacccgacggagtttacgtcatggtcgttttacagagcagggatagcagagtttgtggccacttttctgtttctctacatcactgtcttaaccgttatgggagtcgccggggctaagtctaagtgtagtaccgttgggattcaaggaatcgcttgggccttcggtggcatgatcttcgccctcgtttactgcaccgctggcatctcagggggacacataaacccggcggtgacatttgggctgtttttggcgaggaagttgtcgttgcccagggcgattttctacatcgtgatgcaatgcttgggtgctatttgtggcgctggcgtggtgaagggtttcgaggggaaaacaaaatacggtgcgttgaatggtggtgccaactttgttgcccctggttacaccaagggtgatggtcttggtgctgagattgttggcactttcatccttgtttacaccgttttctccgccaccgatgccaaacgtagcgccagagactcccacgtccccattttggcacccttgccaattgggttcgctgtgttcttggttcacttggcaaccatccccatcaccggaactggtatcaaccctgctcgtagtcttggtgctgctatcatcttcaacaaggaccttggttgggatgaacactggatcttctgggtgggaccattcatcggtgcagctcttgcagcactctaccaccaggtcgtaatcagggccattcccttcaagtccaagtgattcaatcaaacggttcatgcttaatcaagttgggaacaacaacaacaacaaaaatcaagccaatgtttgtgggttttggtttcatttcattaagatgatctgtttatctcttttcttctttttaaaatttaaagtctttgtattttgtatgtaaagatgtaaaattatgattattaggtggtgcatgtgtcgcgtcatgggccaatgttatcctctgcttttaagttggaagaggcccaactcatgtgtgatgtacggctgtgattgtgtaatttaatttgcaaaatcaaaaataacaccagagtcatatatatgcatctctttattttctctggcccccaccatgtcttctatgtaatatttgttgccctcttcccccaagtatatgacaaggttgggtttctttttaaacaaaaaaaaaaaaaaaa

SEQ ID NO: 49 - DNA, 1591 bp, Glycine max
ctcaggctaatcgagctggtactaaactaatgcatattaggtaatgcaaataaataataacgctcccaagaatattcaaatggtttcttttgctttttgcttaacgacttttgtatctctacgtattacttgagaaaaaaagctgctattattatccaactaaacaaatgaaagctacagttaaggacatggcctattaacaatattacgtagacttgatcattgtctcatccacgagatagaaacaaaatatataaaagggctcattatgcttatttagttcatcaagaagctaggaaaatgagtacgtagaatgaacatttaataatggacgtgagagaagttaatcgctgacagccatgtgccgaccatgttttttataaatgaaaagaaagaaatgttcgtatataataattaacggacacaagaaccttgttaataattatcattatctttttttttttgtttttattttccgaaaaacttgtttctccaatcattgatgtgtatttctattctctctccatttccaactcctgactgagaagtggatttcatatcaacattagcaattagtagaatactatcatctttcacgctacaaaacattggtactttggtaggtaaagatttgcaaacacgaataagtaattaagaaaggttcatacacattcaatgattctggattcctaccttacgttatttgtttcgaaatacctagatgagagcatcttgttatttattactacatattaattttccctgtgtaccttgtcgtagtttaaatttattattttttcaatcataaataaatataagaaatatttttttcttaatataattttattttatatttaaaaataaatcataatttgaaagagctacaaatttataccacatgtgggaagtattgttggtttctccaaccatacttattgagaataacttgaatttatattcaacgtattaattgcttcacctttaacgtgccaaaataataataataaaaaacttaaaactactgtattaatcgcgtgtggttgaatggaggcaaattctattctaaaaaagaaaagcattaacaaaaggagaaaagaaaaactgttgacacctgacagcagtaacagggaactgggaagtagcagtaggagtatttgcgtgttggtttccaactctggaatccaccgtgccaaactgcgaatgcaggagaaatcgacacgtgtccatttgcaggcgcgagttgaacgtgacaatgcaccaccgcccagcatcgaacgcagccaaggaccacgtcgaaaccacagtaatccacgttccagtgctgcgcggaacatggtcggtctttctaggagtggttggaatcacgccagctaggacaaaccccatcaatcattggtcattatcaaacaaaacatttcaaaaattcaacatattacgcctcgggacccacctcccactacacctcaccctcacttctattaactcgaacacattcgggttataaatccgcaaccctccttctcactcactcactcactcactcactcactcgcaagcaaaaagaaagaatcccaggcgaggagaaagatgg

SEQ ID NO: 50 - DNA, 65 bp, glycine max
ctcactcactcactcactcactcactcactcgcaagcaaaaagaaagaatcccaggcgaggagaa